SEQUENCE LISTING (4 sequences)

SEQ ID NO: 1 - protein, 1615 aa, synthetic construct
  source 1..1615: note = DNMT1
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

SEQ ID NO: 2 - DNA, 18 bp, synthetic construct
  source 1..18: mol_type = other DNA; note = polynucleotide
  modified_base 3: mod_base = OTHER; note = hydroxymethylcytosine
  modified_base 8: mod_base = OTHER; note = methylcytosine
  misc_binding 3: bound_moiety = quencher
  misc_binding 8: bound_moiety = quencher
tgctgaacatgtagggta

SEQ ID NO: 3 - DNA, 10 bp, synthetic construct
  source 1..10: mol_type = other DNA; note = polynucleotide
  misc_binding 10: bound_moiety = fluorophore
taccctacat

SEQ ID NO: 4 - DNA, 15 bp, synthetic construct
  source 1..15: mol_type = other DNA; note = polynucleotide
  misc_binding 15: bound_moiety = fluorophore
taccctacatgttca

(19) United States
(12) Patent Application Publication
(10) Pub. No.: US 2026/0297654 A1
(43) Pub. Date: Oct. 1, 2026

(54) DETECTING A METHYLATED NUCLEOTIDE USING A QUENCHER COUPLED TO THE METHYLATED NUCLEOTIDE

(71) Applicant: Illumina, Inc., San Diego, CA (US)

(72) Inventors: Rebekah Karadeema, San Diego, CA (US); Gaetano Speciale, San Diego, CA (US)

(73) Assignee: Illumina, Inc., San Diego, CA (US)

(21) Appl. No.: 19/482,204

(22) PCT Filed: May 13, 2024
(86) PCT No.: PCT/US2024/029126

§ 371 Date: Nov. 6, 2025

Related U.S. Application Data
(60) Provisional application No. 63/505,258, filed on May 31, 2023.

(51) Int. Cl. C12Q 1/6818

(52) U.S. Cl. CPC C12Q 1/6818

(57) ABSTRACT

Examples provided herein are related to detecting a methylated nucleotide using a quencher coupled to the methylated nucleotide. In some examples, a methylated nucleotide in a polynucleotide may be detected using a method that includes coupling a quencher to the methylated nucleotide. The method may include adding fluorescently labeled nucleotides to a primer hybridized to the polynucleotide. The method may include using the quencher to reduce fluorescence from at least one of the added, fluorescently labeled nucleotides. The method may include using the reduced fluorescence caused by the first quencher to detect the methylated nucleotide.

### CROSS-REFERENCE TO RELATED APPLICATIONS

This application claims the benefit of U.S. Provisional Patent Application No. 63/505,258, filed May 31, 2023 and entitled “Detecting a Methylated Nucleotide Using a Quencher Coupled to the Methylated Nucleotide,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2313-PCT.xml”, was created on Apr. 26, 2024 and is 12 kB in size.

### FIELD

This application relates to methods for detecting methylated nucleotides.

### BACKGROUND

Within living organisms, such as humans, selected cytosines (Cs) in the genome may become methylated. For example, S-adenosyl-L-methionine (SAM) is known to be a ubiquitous methyl donor for a variety of biological methylation reactions that are catalyzed by enzymes referred to as methyltransferases (MTases). The enzyme 5-MTase may add a methyl group to the 5-position of cytosine to form 5-methylcytosine (5-mC) in a manner such as described in Deen et al., “Methyltransferase-directed labeling of biomolecules and its applications,” Angewandte Chemie International Edition 56:5182-5200 (2017), the entire contents of which are incorporated by reference herein. Other enzyme(s) may oxidize the cytosine's methyl group to form the 5-mC derivative 5-hydroxymethylcytosine (5-hmC), and may oxidize the 5-hmC further to form the 5-mC derivatives 5-formylcytosine (5-fC) and 5-carboxylcytosine (5-caC).

5-mC and 5-hmC may be referred to as epigenetic markers, and it can be desirable to detect them in a genomic sequence. For example, 5-mC is proposed to have diverse roles in regulation of gene expression, parental imprinting, and molecular etiology of human diseases including cancer. Hundreds of methylation biomarkers have been discovered for cancer and other diseases, and methylation signatures in circulating cell-free DNA (cfDNA) have shown promise as a basis for liquid biopsy assays for diagnoses, treatment selection, and disease monitoring.

Two broad categories of approaches have been developed to measure DNA methylation. Enrichment strategies select methylated DNA fragments using a 5-mC-specific antibody, methylation-sensitive restriction enzymes, or methylation-induced changes in DNA duplex stability. The methylated DNA fragments then can be measured in relation to a non-enriched sample by qPCR or other standard nucleic acid quantitation strategies. Methylation assays based on chemical transformation begin by treating the sample with a chemical or enzymatic reagent that creates a difference in base pairing between methylated and non-methylated cytosine residues. The current golden standard method for detecting 5-mC and 5-hmC is bisulfite sequencing, which converts any unmethylated C in the sequence to uracil (U), but does not convert 5-mC or 5-hmC to the corresponding uracil derivatives. When the sequence is amplified using polymerase chain reaction (PCR), the uracil is amplified as thymidine (T), and as such the unmethylated C is sequenced as T. In comparison, the 5-mC and 5-hmC are amplified as C, and as such are sequenced as C. Thus, any Cs in the sequence may be identified as corresponding to 5-mC or 5-hmC because they had not been converted to U. Such a scheme may be referred to as a “three-base” sequencing scheme because any unmethylated C is converted to T. However, this type of scheme reduces sequence complexity and may lead to reduced sequencing quality, lower mapping rates, and relatively uneven coverage of the sequence.

Despite the importance of DNA methylation in the etiology of many human diseases, and the identification of hundreds of methylation biomarkers for cancer and other disorders, only a small number of methylation-based diagnostic assays have been adopted for use in the clinic. A major reason for this discrepancy is the relative difficulty of measuring cytosine methylation as compared to SNPs and other DNA sequence changes. Cytosine methylation is a relatively minor chemical change in the structure of the nucleobase, and on its own does not change the pattern of hydrogen bond donors and acceptors that govern specific base pairing.

### SUMMARY

Examples provided herein are related to detecting a methylated nucleotide using a quencher coupled to the methylated nucleotide. Compositions and methods for performing such detection are disclosed.

Some examples herein provide a method for detecting a first methylated nucleotide in a polynucleotide. The method may include coupling a first quencher to the first methylated nucleotide. The method may include adding fluorescently labeled nucleotides and a polymerase to a primer hybridized to the polynucleotide. The method may include using the first quencher to reduce fluorescence from at least one of the added, fluorescently labeled nucleotides. The method may include using the reduced fluorescence caused by the first quencher to detect the first methylated nucleotide.

In some examples, the first methylated nucleotide is selected from the group consisting of 5-methylcytosine (5-mC), 5-hydroxymethylcytosine (5-hmC), 5-formylcytosine (5-fC), 5-carboxylcytosine (5-caC) and 6-methyladenine (6-mA).

In some examples, coupling the first quencher to the first methylated nucleotide includes: oxidizing the 5-mC, 5-hmC, or 5-fC to 5-carboxylcytosine (5-caC); reacting the 5-carboxyl group with a first molecule to form a first product. In some examples, the first product includes the first quencher. In some examples, the method further includes coupling the first quencher to the first product.

In some examples, a ten-eleven translocation (TET) dioxygenase is used to oxidize the 5-mC, 5-hmC, or 5-fC to 5-caC. In some examples, a chemical reagent is used to oxidize the 5-mC, 5-hmC, or 5-fC.

In some examples, the method further includes activating the 5-carboxyl group of the 5-caC before reacting the 5-carboxyl group with the first molecule. In some examples, the 5-carboxyl group of the 5-caC is activated using 4-(4,6-dimethoxy-1,3,5-triazin-2-yl)-4-methyl-morpholinium chloride (DMTMM) or 1-ethyl-3-(3′-(dimethylamino) propyl) carbodiimide (EDC). In some examples, the first molecule includes a nucleophile. In some examples, the first molecule includes an azirine.

In some examples, the first methylated nucleotide is 6-mA.

In some examples, the first methylated nucleotide is 5-mC. In some examples, coupling the first quencher to the first methylated nucleotide includes: reacting the 5-methyl group of the 5-mC with a first molecule to form a first product; and reacting the first product with a second molecule to couple the first quencher to the first product. In some examples, reacting the methyl group of the 5-mC with the first molecule includes using CMD1 to couple the first molecule to the 5-methyl group. In some examples, the first product includes a diol. In some examples, the second molecule includes a boronate.

In some examples, coupling the first quencher to the first methylated nucleotide includes: oxidizing the 5-mC to 5-hmC; and reacting the 5-hydroxymethyl group of the 5-hmC with a first molecule to form a first product; and reacting the first product with a second molecule to couple the first quencher to the first product. In some examples, a ten-eleven translocation (TET) dioxygenase is used to oxidize the 5-mC to the 5-hmC. In some examples, the TET dioxygenase includes ccTET. In some examples, reacting the hydroxymethyl group of the 5-hmC with the first molecule includes using Mha.I to couple the first molecule to the hydroxymethyl group. In some examples, the first molecule includes an aminothiol. In some examples, the second molecule includes an N-hydroxysuccinimide (NHS) ester, an isocyanate, or an isothiocyanate.

In some examples, the first methylated nucleotide is 5-hmC. In some examples, coupling the quencher to the first methylated nucleotide includes: reacting the 5-hydroxymethyl group of the 5-hmC with a first molecule to form a first product; and reacting the first product with a second molecule to couple the first quencher to the first product. In some examples, reacting the hydroxymethyl group of the 5-hmC with the first molecule includes using Mha.I to couple the first molecule to the hydroxymethyl group. In some examples, the first molecule includes an aminothiol. In some examples, the second molecule includes an N-hydroxysuccinimide (NHS) ester, an isocyanate, or an isothiocyanate.

In some examples, the polynucleotide includes a second methylated nucleotide, and the method further may include: coupling a second quencher to the second methylated nucleotide; using the second quencher to reduce fluorescence from at least one of the added, fluorescently labeled nucleotides; and using the reduced fluorescence caused by the second quencher to detect the second methylated nucleotide.

In some examples, the polynucleotide is coupled to a substrate. In some examples, the polynucleotide is within a cluster of polynucleotide amplicons coupled to the substrate. In some examples, the polynucleotide amplicons of the cluster also respectively include first methylated nucleotides, and the method further may include coupling the first quencher to the first methylated nucleotides of the respective amplicons; adding fluorescently labeled nucleotides to primers respectively hybridized to the amplicons; using the first quencher to reduce fluorescence from at least one of the added, fluorescently labeled nucleotides; and using the reduced fluorescence caused by the first quencher to detect the first methylated nucleotides in the respective amplicons.

Some examples herein provide an isolated polynucleotide from an extracellular fluid sample. The polynucleotide may include a methylated nucleotide coupled to a quencher.

In some examples, the methylated nucleotide is directly coupled to the quencher. In some examples, the methylated nucleotide is indirectly coupled to the quencher.

In some examples, the methylated nucleotide is selected from the group consisting of: 5-methylcytosine (5-mC), 5-hydroxymethylcytosine (5-hmC), 5-formylcytosine (5-fC), 5-carboxylcytosine (5-caC), and 6-methyladenine (6-mA).

In some examples, the methylated nucleotide is 5-caC. In some examples, the 5-caC is coupled to the quencher via a carboxyl group of the 5-caC. In some examples, the 5-caC is coupled to the quencher via an azirine.

In some examples, the methylated nucleotide is 6-mA.

In some examples, the methylated nucleotide is 5-mC. In some examples, the 5-mC is coupled to the quencher via a diol. In some examples, the 5-mC is coupled to the quencher via a boronate.

In some examples, the methylated nucleotide is 5-hmC. In some examples, the 5-hmC is coupled to the quencher via an aminothiol. In some examples, the 5-hmC is coupled to the quencher via an N-hydroxysuccinimide (NHS) ester, an isocyanate, or an isothiocyanate.

In some examples, the polynucleotide is coupled to a substrate. In some examples, the polynucleotide is within a cluster of polynucleotide amplicons coupled to the substrate. In some examples, the polynucleotide amplicons of the cluster also include first methylated nucleotides and quenchers respectively coupled thereto.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1H schematically illustrate example compositions and operations in a process flow for detecting a methylated nucleotide using a quencher coupled to the methylated nucleotide.

FIGS. 2A-2C illustrate plots of example fluorescence intensity as a function of the number of fluorescent nucleotides added to a primer hybridized to a polynucleotide including a methylated nucleotide having a quencher coupled thereto, for different types of systems.

FIGS. 3A-3E schematically illustrate example compositions and operations in a process flow for detecting different methylated nucleotides using different quenchers respectively coupled to the different methylated nucleotides.

FIG. 4 illustrates a plot of example fluorescence intensities as a function of the number of fluorescent nucleotides added to a primer hybridized to a polynucleotide including different methylated nucleotides having different quenchers respectively coupled thereto.

FIG. 5 schematically illustrates example compositions and operations in a process flow for detecting methylated nucleotides within a cluster of amplicons.

FIG. 6 illustrates a flow of operations in an example method for detecting a methylated nucleotide using a quencher coupled to the methylated nucleotide.

FIGS. 7A-7F schematically illustrate example compositions and operations in a process flow for producing clonal clusters that preserve the CpG methylation state of a target polynucleotide.

FIG. 8 shows an example sequence of the human template-dependent DNA (cytosine-5)-methyltransferase 1 (DNMT1, SEQ ID NO:1).

### DETAILED DESCRIPTION

Examples provided herein are related to detecting a methylated nucleotide using a quencher coupled to the methylated nucleotide. Compositions and methods for performing such detection are disclosed.

Provided herein is detection of nucleotide methylation in which a quencher coupled to a methylated nucleotide generates a signal indicative of the methylated nucleotide. In a manner such as described in greater detail below, the quencher may be coupled to any of a variety of methylated nucleotides that may occur within a polynucleotide. Such methylated nucleotide may be naturally occurring within the polynucleotide, or may be the product of a reaction with a nucleotide within the polynucleotide. A primer may be hybridized to the polynucleotide, and the primer may be extended using a polymerase to add fluorescently labeled nucleotides. As different fluorescently labeled nucleotides are added to the primer based on the sequence of the polynucleotide, some of the nucleotides may be relatively closer to the quencher, and the quencher may inhibit the fluorescence from such nucleotides relatively strongly; while others of the nucleotides may be relatively farther from the quencher, and the quencher may inhibit the fluorescence from such nucleotides relatively weakly, if at all. Accordingly, the intensity of the fluorescent signal from the nucleotides being added may have relatively high level at locations that are sufficiently spatially separated from the quencher, may decrease to a minimum at a location adjacent to the quencher. Additionally, in some examples, the fluorescent signals from the various nucleotides being added may be used to determine the sequence of the polynucleotide, e.g., using sequencing-by-synthesis as is known in the art. As such, the location of the quencher (that is, where the signal may be most reduced) may be correlated to the location and identity of the methylated nucleotide at which the signal is most reduced.

In some examples, the present methylation detection may be performed directly on the sequencer without the need for user-based library preparation, which has traditionally used chemistry to convert either mC or unmethylated C to T, which otherwise may result in difficulties in read mapping such as explained above. The present methylation detection is compatible, among other things, with PCR-free library preparations.

First, some terms used herein will be briefly explained. Then, some example compositions and example methods will be described for detecting a methylated nucleotide using a quencher coupled to the methylated nucleotide.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another. When the first and second polynucleotide are hybridized to one another, pairs of bases may be “opposite” to each other, and the bases of that pair may be said to “associate” with each other. When bases of a given pair are complementary to each other, those bases also may be said to “hybridize” to one another. On the other hand, when one base of a given pair is pulled away from the other base of that pair, the bases may be said to “disassociate” from each other.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue (also referred to as a modified base) which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. Other modified bases may include targets and/or fluorophores in a manner such as described elsewhere herein. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate. A “capture primer” refers to a primer that may be used to seed and/or to amplify a polynucleotide that includes an adapter which is substantially complementary to the capture primer.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, CA).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylated nucleotide” refers to a nucleotide that includes a methyl group (—CH₃ or Me) or a derivatized methyl group. For example, as used herein, the term “methylcytosine” or “mC” refers to cytosine in DNA (namely, 2′-deoxycytosine) that includes a methyl group (—CH₃ or Me), or a derivative of methylcytosine. As another example, “methyladenine” or “mA” refers to adenine that includes a methyl group, or is a derivative of methyladenine. As used herein, a “derivative” of a methylated nucleotide (such as a derivative of methylcytosine or a derivative of methyladenine”) refers to a nucleotide having a methyl group or a derivatized methyl group. A nonlimiting example of a derivatized methyl group is an oxidized methyl group. A nonlimiting example of an oxidized methyl group is hydroxymethyl (—CH₂OH). An mC derivative having a hydroxymethyl group may be referred to as hydroxymethylcytosine or hmC. Another nonlimiting example of an oxidized methyl group is formyl group (—CHO). An mC derivative having a formyl group may be referred to as formylcytosine or fC. Another nonlimiting example of an oxidized methyl group is carboxyl (—COOH). An mC derivative having a carboxyl group may be referred to as carboxylcytosine or caC. The methyl group may be located at the 5 position of the cytosine, in which case the mC may be referred to as 5-mC. The oxidized methyl group may be located at the 5 position of the cytosine, in which case the hmC may be referred to as 5-hmC, the fC may be referred to as 5-fC, or the caC may be referred to as 5-caC. The methyl group of methyladenine may be located at the 6 position of the adenine, in which case the mA may be referred to as 6 mA.

As used herein, the term “fluorophore” is intended to mean an element that emits light at a first wavelength (“emission,” or “fluorescence”) responsive to excitation with light at a second wavelength (“optical excitation,” or “excitation light”) that is different from the first wavelength. The light emitted by a fluorophore may be referred to as “fluorescence” and may be detected by suitable optical circuitry. The light emitted by a fluorophore may have an “emission lifetime” that characterizes the intensity as a function of time with which the fluorophore fluoresces after optical excitation. In various examples, a fluorophore may be or include a molecule such as an organic dye, a fluorescent protein, or a particle such as a quantum dot. Example organic dyes include xanthene derivatives (such as fluorescein and rhodamine and their derivatives), cyanine and its derivatives, squaraine derivatives and ring-substituted squaraines, squaraine rotaxane derivatives, naphthalene derivatives, coumarin derivatives, oxadiazole derivatives, anthracene derivatives, pyrene derivatives, oxazine derivatives, acridine derivatives, arylmethine derivatives, tetrapyrrole derivatives, and dipyrromethene derivatives. Example fluorescent proteins include green fluorescent protein (GFP), yellow fluorescent protein (YFP), and red fluorescent protein (RFP). Some specific, nonlimiting examples of organic dyes that may be used as fluorophores include rhodamine and its derivatives such as TMR (tetramethylrhodamine), TAMRA (carboxytetramethylrhodamine), or 5TAMRA (5-carboxytetramethylrhodamine); fluorescein and its derivatives such as FAM (fluorescein amidite) or 5FAM (5-carboxyfluorescein); cyanine and its derivatives such as cyanine3 (Cy3,1-[6-(6-aminohexylamino)-6-oxohexyl]-3,3-dimethyl-2-[(1E,3E)-3-(1,3,3-trimethylindolin-2-ylidene) prop-1-enyl]-3H-indolium chloride hydrochloride) or cyanine5 (Cy5, 1-[6-(2,5-dioxopyrrolidin-1-yloxy)-6-oxohexyl]-3,3-dimethyl-2-[(1E,3E,5E)-5-(1,3,3-trimethylindolin-2-ylidene) penta-1,3-dienyl]-3H-indolium tetrafluoroborate); BODIPY (4,4-difluoro-4-bora-3a,4a-diaza-s-indacene); Atto dyes commercially available from Millipore Sigma; XL665 commercially available from CisBio (phycobilliprotein pigment purified from red algae); or Alexa Fluor dyes commercially available from ThermoFisher Scientific, such as Alexa Fluor 680, Alexa Fluor 488, Alexa Fluor 546, Alexa Fluor 633, and Alexa Fluor 647. In still other examples, the fluorophore may include a quantum dot.

By “quantum dot” it is meant a particle including about 100 to about 100,000 atoms and a diameter of about 2 to about 10 nm, and that emits light in response to excitation light or energy transfer. Quantum dots may include, or may consist essentially of, inorganic atoms. Quantum dots may include atoms from groups II-IV, groups III-V, or groups IV-VI of the period table, and may include a core having a first composition that is covered by a shell having a second, different composition. Cadmium (Cd) may be included in the core and/or in the shell. In one nonlimiting example, a quantum dot includes a CdSe core covered by a ZnS shell, and may be referred to as a CdSe/ZnS core/shell quantum dot. In another nonlimiting example, a quantum dot includes a CdSe core covered by a CdS shell, and may be referred to as a CdSe/ZnS core/shell quantum dot. Quantum dots may have relatively narrow emission peaks, and may have a brighter emission and a higher signal to noise ratio as compared to organic dyes (e.g., may be about 10-20 times brighter than organic dyes). Quantum dots also may be relatively stable because their inorganic composition may inhibit the effect of photobleaching. Quantum dots also may have a significantly longer fluorescence time (e.g., about 10-40 ns) as compared to that of inorganic dyes (e.g., a few nanoseconds).

As used herein, the term “quencher” is intended to mean an element that, when in sufficient proximity to a fluorophore, reduces or inhibits fluorescence from that fluorophore. In various examples, a quencher may be or include a molecule. Example commercially available quenchers include DABCYL (dimethylaminoazobenzenesulfonic acid, Jena Bioscience GMBH), Black Hole Quencher dyes (BHQ, LGC Biosearch Technologies), IOWA BLACK® FQ (Integrated DNA Technologies), IOWA BLACK® RQ (Integrated DNA Technologies), and IRDYE® QC-1 (LI-COR Biosciences).

As used herein, to “detect” fluorescence is intended to mean to receive light from a fluorophore, to generate an electrical signal based on the received light, and to determine, using the electrical signal, that light was received from the fluorophore. Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore is present.

To “measure” fluorescence is intended to mean to determine a relative or absolute amount of the fluorescence that is detected. For example, the amount of fluorescence may be measured relative to a baseline amount of fluorescence, or as an absolute amount of fluorescence. Illustratively, the amount of fluorescence from one or more fluorophores may be correlated to the amount of a modified base, in a polynucleotide, that is hybridized to the methylated nucleotide. For example, the memory of the electronic circuitry described above may store instructions causing the processor to monitor the level of the electrical signal at one or more times, and to correlate such level(s) to an amount of the methylated nucleotide.

### Compositions and Methods for Detecting a Methylated Nucleotide Using a Quencher Coupled to the Methylated Nucleotide

Provided herein are example assays for detecting methylated nucleotides that may be used for targeted, highly multiplexed, quantitative measurement of methylated nucleotides at any respective location(s) within a target polynucleotide. More specifically, quenchers respectively may be coupled to suitable methylated nucleotides within the target polynucleotide, and the presence of the methylated nucleotides may be detected via the quenchers' reducing fluorescence intensity from fluorescently labeled nucleotides that are added to a primer hybridized to the target polynucleotide. In some examples, multiple different types of quenchers respectively may be coupled to different types of methylated nucleotides, thus providing for a wide variety of epigenetic assays to be performed concurrently with one another using the same or similar workflow as may be used to sequence the target polynucleotide itself.

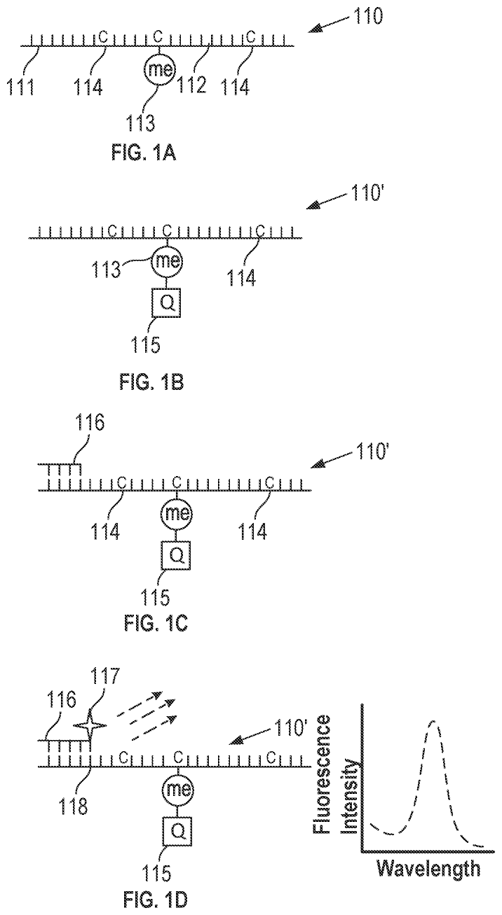

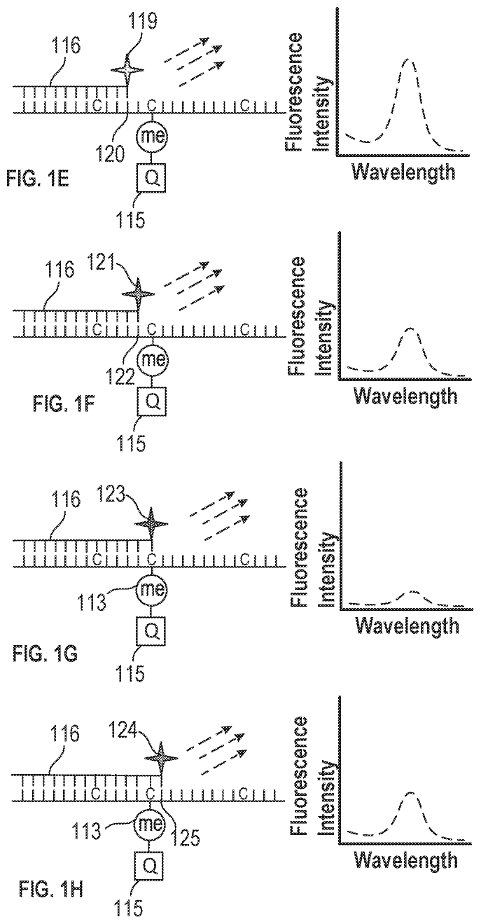

FIGS. 1A-1H schematically illustrate example compositions and operations in a process flow for detecting a methylated nucleotide using a quencher coupled to the methylated nucleotide. The composition illustrated in FIG. 1A includes target polynucleotide 110, e.g., a fragment of single-stranded DNA or RNA. Polynucleotide 110 may include sugar-phosphate backbone 111 and bases 112. It will be appreciated that polynucleotide 110 may be significantly longer than is suggested in FIG. 1A, and that the polynucleotide may be in any suitable fluid or may be coupled to a substrate. It may be desired to assay whether any locations within polynucleotide 110 may include one or more methylated nucleotides. In the nonlimiting example illustrated in FIG. 1A, polynucleotide 110 includes a methylated nucleotide 113, and a plurality of cytosines 114, in addition to other bases the types of which are not specifically illustrated. For simplicity, polynucleotide 110 is illustrated as including a single methylated nucleotide 113. However, it will be appreciated that polynucleotide 110 may include a plurality of methylated nucleotides of the same type as one another or of different types. In some examples, methylated nucleotide 113 may be selected from the group consisting of mC, hmC, fC, caC, and 6 mA.

In a manner such as provided herein, a quencher may be coupled to a methylated nucleotide 113 and used in such a manner that the methylated nucleotide 113 may be distinguished from other nucleotides. In the nonlimiting example illustrated in FIG. 1A, a quencher may be coupled to methylcytosine 113 and used in such a manner that the methylcytosine 113 may be distinguished from cytosines 114. In other examples (not specifically illustrated), a quencher may be coupled to 6 mA and used in such a manner that the 6 mA may be distinguished from adenines in the polynucleotide. For example, FIG. 1B illustrates polynucleotide 110′ which includes a methylated nucleotide 113 (e.g., 5-mC, 5-hmC, 5-fC, 5-caC, or 6-mA) coupled to quencher 115. In comparison, quenchers are not coupled to cytosines 114 (or other unmethylated nucleotides). In some examples, methylated nucleotide 113 may be directly coupled to the quencher 115, while in other examples, the methylated nucleotide is indirectly coupled to the quencher. In either circumstance, quencher 115 selectively may be coupled to the methylated nucleotide 113 as compared to the unmethylated nucleotides 114. Nonlimiting examples of methylated nucleotides, and nonlimiting manners of selectively coupling quenchers to such nucleotides, are described in greater detail below. Additionally, any suitable number of different quenchers selectively may be coupled to any suitable number of different methylated nucleotides and used to detect such nucleotides in a manner such as described in greater detail below.

As illustrated in FIG. 1C, a primer 116 may be hybridized to polynucleotide 110′. The primer may be extended using a plurality of fluorescently labeled nucleotides, and the sequence of nucleotides added to the primer is complementary to the sequence of nucleotides in polynucleotide 110′. The respective colors of fluorescence from the labels of such nucleotides may be used to determine the sequence of polynucleotide 110′, e.g., using a one-channel, two-channel, or four-channel process similar to that known in the art. However, whereas previously known processes may determine only the sequence of the target polynucleotide, the present processes also may be used to detect the presence of methylated nucleotides within the target polynucleotide.

For example, at the particular time illustrated in FIG. 1D, a polymerase (not specifically illustrated) has added a first fluorescently labeled nucleotide 117 to the 3′ end of primer 116 that is complementary to nucleotide 118 within polynucleotide 110′. The fluorophore of nucleotide 117 is excited, and responsive to such excitation emits fluorescence at a particular wavelength and intensity, as intended to be suggested by the plot illustrated in FIG. 1D. Suitable optical detection circuitry (not specifically illustrated) detects the fluorescence from the fluorophore of nucleotide 117. The identity of nucleotide 117, and thus the identity of complementary nucleotide 118, may be determined from the wavelength of the fluorescence in a manner such as known in the art. Additionally, as provided herein, information about the distance of nucleotide 117 from quencher 115, and thus information about the distance of nucleotide 118 from quencher 115, may be determined from the intensity of the fluorescence. In the nonlimiting example illustrated in FIG. 1D, nucleotide 117 is sufficiently far from quencher 115 that the intensity of fluorescence from the label of such nucleotide is substantially not affected by the presence of quencher 115. That is, the intensity of fluorescence from labeled nucleotide 117 may be substantially the same as it would be if polynucleotide 110′ was not coupled to any quenchers.

However, as provided herein, as primer 116 is further extended using a polymerase to add additional fluorescently labeled nucleotides to the 3′-end of the primer, some of the nucleotides may be added to a location that is sufficiently close to quencher 115 that quencher may reduce fluorescence from such nucleotides. Additionally, the amount by which quencher 115 reduces the fluorescence from such nucleotides may be used to detect methylated nucleotide 113. For example, as illustrated in FIG. 1E, fluorescence from labeled nucleotide 119, which is complementary to and added opposite to nucleotide 120, may be reduced compared to the fluorescence from labeled nucleotide 117 because nucleotide 119 is closer to quencher 115 than is nucleotide 117. Such reduction in fluorescence is intended to be suggested by the darkened shading of nucleotide 119 and the reduced intensity of fluorescence illustrated in the plot of FIG. 1E. The identity of nucleotide 119, and thus the identity of complementary nucleotide 120, may be determined from the wavelength of the fluorescence in a manner such as known in the art.

Additionally, as illustrated in FIG. 1F, fluorescence from labeled nucleotide 121, which is complementary to and added opposite to nucleotide 122, may be reduced compared to the fluorescence from labeled nucleotide 119 because nucleotide 121 is even closer to quencher 115 than is nucleotide 119. Such reduction in fluorescence is intended to be suggested by the further darkened shading of nucleotide 121 and the further reduced intensity of fluorescence illustrated in the plot of FIG. 1F. The identity of nucleotide 121, and thus the identity of complementary nucleotide 122, may be determined from the wavelength of the fluorescence in a manner such as known in the art. Additionally, as illustrated in FIG. 1G, fluorescence from labeled nucleotide 123, which is complementary to and added opposite to nucleotide 113, may be reduced compared to the fluorescence from labeled nucleotide 121 because nucleotide 123 is even closer to quencher 115 than is nucleotide 121. Such reduction in fluorescence is intended to be suggested by the further darkened shading of nucleotide 123 and the further reduced intensity of fluorescence illustrated in the plot of FIG. 1G. In this regard, because labeled nucleotide 123 is directly opposite the methylated nucleotide 113 to which quencher 115 is coupled, fluorescence from labeled nucleotide 123 may be expected to be reduced more strongly than the fluorescence from labeled nucleotides that are added further away from the quencher. The identity of nucleotide 123, and thus the identity of complementary nucleotide 113 (that is, methylated nucleotide 113), may be determined from the wavelength of the fluorescence in a manner such as known in the art.

As still further labeled nucleotides are added to primer 116, the fluorescence from such nucleotides may increase with their distance from methylated nucleotide 113, and thus from quencher 115. For example, as illustrated in FIG. 1H, fluorescence from labeled nucleotide 124, which is complementary to and added opposite to nucleotide 125, may be increased compared to the fluorescence from labeled nucleotide 123 because nucleotide 124 is farther from quencher 115 than is nucleotide 123. Such reduction in fluorescence is intended to be suggested by the lightened shading of nucleotide 124 and the increased intensity fluorescence illustrated in the plot of FIG. 1H. The identity of nucleotide 124, and thus the identity of complementary nucleotide 125, may be determined from the wavelength of the fluorescence in a manner such as known in the art. In some examples, labeled nucleotide 124 may be spaced by a similar distance from quencher 115 as is nucleotide 121 (e.g., both nucleotide 121 and nucleotide 124 may be located opposite the nucleotides which are directly adjacent to methylated nucleotide 113). Similarly, fluorescence from labeled nucleotides may further increase as a function of distance from quencher 115.

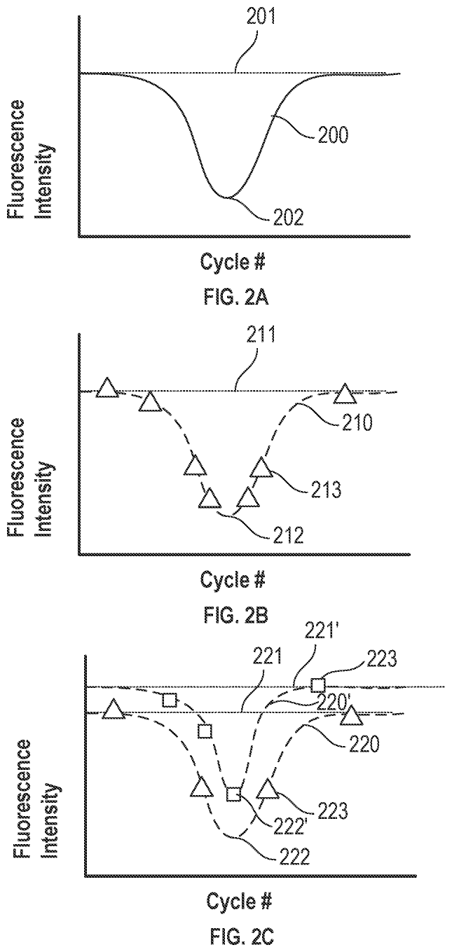

Methylated nucleotide 113 may be detected using the reduced fluorescence caused by the first quencher. For example, FIG. 2A illustrates a plot of example fluorescence intensity 200 as a function of the number of fluorescent nucleotides added to a primer hybridized to a polynucleotide including a methylated nucleotide having a quencher coupled thereto. Each “cycle” referred to in FIG. 2A corresponds to addition of a single nucleotide to primer 116. As such, FIG. 2A illustrates one example relationship between the intensity of fluorescence from different fluorescently labeled nucleotides (e.g., nucleotides 117, 119, 121, 123, 124 respectively described with reference to FIGS. 1D-1H), and the respective distances of such nucleotides from quencher 114. From FIG. 2A, it may be understood that nucleotides that are sufficiently far from quencher 115 (that is, nucleotides added in cycles that are relatively low or relatively high in number relative to the cycle in which nucleotide 123 is added opposite methylated nucleotide 113), may fluoresce with an intensity that is substantially unaffected by the presence of quencher, e.g., may have a fluorescence intensity at approximately baseline 210 illustrated in FIG. 2A. In comparison, nucleotides that are sufficiently close to quencher may fluoresce with intensities that generally decrease as a function of distance from the quencher (that is, nucleotides added in cycles that are sufficiently close to the cycle in which labeled nucleotide 123 is added opposite methylated nucleotide 113). As discussed above with reference to FIG. 1G, the fluorescence from labeled nucleotide 123, added opposite methylated nucleotide 113 may be expected to be the lowest of all the labeled nucleotides because it is the closest to quencher 115. Minimum 202 in FIG. 2A is intended to correspond to the fluorescence intensity from labeled nucleotide 123, and it may be seen that the fluorescence intensity 200 increases on either side of such minimum.

Although FIG. 2A may suggest that the fluorescence intensity 200 varies smoothly as a function of cycle number, in an actual implementation the fluorescence intensity may vary in a more complicated manner. For example, cycle numbers have integer values, and thus the fluorescence intensity may vary in a stepwise manner from one cycle to another rather than as a continuum as is suggested in FIG. 2A. Additionally, the fluorescence intensity may vary as a function of the particular fluorophore(s) that respectively are coupled to different nucleotides, because different fluorophores may interact differently than one another with a given quencher. Additionally, some nucleotides may never be coupled to a fluorophore, and instead are detected through the absence of fluorescence; illustratively, in some commercial instruments G is “dark” because it is not coupled to a fluorophore, and is detected through an absence of fluorescence.

Illustratively, in a one-channel system such as known in the art, only a single type of fluorophore may be used, and such fluorophore may be coupled to different types of nucleotides at different times than one another using chemistry. For example, the fluorophore may be coupled to A and T at a first time during which detection circuitry obtains a first image, then the fluorophore may be chemically cleaved from A and chemically coupled to C and the detection circuitry may obtain a second image at a second time. If the first image and not the second image include fluorescence from the label, then the nucleotide is identified as A; if the second image and not the first image include fluorescence from the label, then the nucleotide is identified as C; if both the first image and second image include fluorescence from the label, then the nucleotide is identified as T; and if neither the first image nor the second image includes fluorescence from the label, then the nucleotide is identified as G.

In examples in which the present quencher 115 is coupled to methylated nucleotides in a one-channel system, it may be expected that regardless of the particular type of fluorophore that is used, the fluorophores attached to the various nucleotides may physically and chemically interact in a similar manner with quencher 115. Alternatively, the fluorophores may be of different types than one another (e.g., in the case of a two-channel or four-channel system), but may interact similarly as one another with the quencher 115. In either circumstance, it may be expected that fluorescence from each fluorescent label may similarly decrease as a function of proximity to quencher 115. FIG. 2B illustrates a plot of example fluorescence intensity 213 as a function of the number of fluorescent nucleotides added to a primer hybridized to a polynucleotide including a methylated nucleotide having a quencher coupled thereto, for an example one-channel system in which A, T, and C are coupled to the same type of fluorophore as one another at different times than one another, while G is not coupled to any fluorophores. The fluorescence intensities may generally follow the same overall trend 210 as a function of cycle number as does intensity 200 described with reference to FIG. 2A, and the intensities may be approximately at baseline 211 when the fluorophores are sufficiently far from quencher 115. However, the fluorescence intensity 213 measured during a given cycle may be discontinuous with fluorescence intensities measured during the previous and subsequent cycles. For example, when G (to which no fluorophore is coupled) is added, the detection circuitry may not detect any fluorescence-including at minimum 212 of trend 210, where G is added opposite to the methylated nucleotide in a manner such as described with reference to FIG. 1G. A similar trend 210 may be observed in two-channel or four-channel system, in which there are different types of fluorophores which all interact with the quencher similarly as one another.

In a two-channel or four-channel system, different nucleotides may be coupled to different fluorophores than one another, and such fluorophores may emit fluorescence at different wavelengths from which the identities of the nucleotides respectively may be determined. In examples in which the present quencher 115 is coupled to methylated nucleotides in a two-channel system or four-channel system, it may be expected that different types of fluorophores respectively attached to the various nucleotides may physically and chemically interact in different manners with quencher 115. Accordingly, while it may be expected that fluorescence from a given type of label may similarly decrease as a function of proximity to quencher 115 in a manner such as described with reference to FIG. 2B, fluorescence from other types of labels may decrease as a different function of proximity to quencher 115. FIG. 2C illustrates plot of example fluorescence intensity 223 from a first type of fluorophore, and fluorescence intensity 223′ from a second type of fluorophore, as a function of the number of fluorescent nucleotides added to a primer hybridized to a polynucleotide including a methylated nucleotide having a quencher coupled thereto, for an example two-channel or four-channel system in which A is coupled to the first type of fluorophore and G is coupled to the second time of fluorophore.

The fluorescence intensities from the first and second fluorophores respectively may generally follow different overall trends 220, 220′ as a function of cycle number, similarly as does intensity 200 described with reference to FIG. 2A. However, the trend 220 may be a different function of cycle number than is trend 220′, because the first fluorophore may interact differently with quencher 115 than does the second fluorophore. Furthermore, the fluorescence intensity 223 or 223′ measured during a given cycle may be discontinuous with fluorescence intensities measured during the previous and subsequent cycles, and may have different magnitudes because the fluorophores have different intrinsic fluorescent intensities than one another, as reflected by the different baselines 221, 221′ for the two fluorophores, and/or may have different responses to proximity to the quencher than one another. For example, when G (to which the second fluorophore is coupled) is added opposite to the methylated nucleotide in a manner such as described with reference to FIG. 1G, the detection circuitry may detect a level of fluorescence at minimum 222′ of trend 220. Illustratively, and depending on the particular fluorophores that are used, the minimum 222 of trend 220 may have a different value than minimum 222′, and as such one or more of the fluorescence intensities 223 may in some circumstances be approximately the same as intensity 223′ at minimum 222′.

For examples such as described with reference to FIGS. 2A-2C, appropriate processing circuitry may be used to determine the location of the minimum and thus to determine the location of the methylated nucleotide to which the quencher is coupled. For example, a base calling algorithm may be generated by using the present operations to sequence known DNA sequences including methylated nucleotides respectively coupled to quenchers (e.g., within any suitable number of one or more CpG sites, where “CpG” refers to a region of a polynucleotide in which C is followed by G in the 5′ to 3′ direction). The measured reductions in fluorescence caused by the quenchers may be recorded, and bioinformatically used to create a base calling algorithm that may be used to detect similar reductions in fluorescence and correlate such reductions to the respective presence of methylated nucleotides coupled to quenchers.

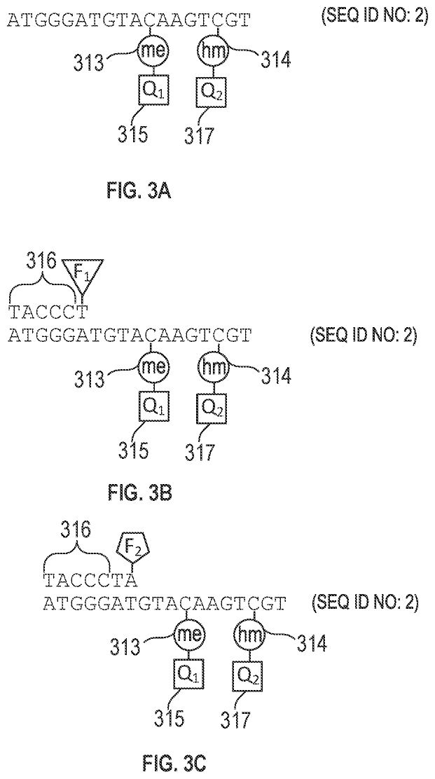

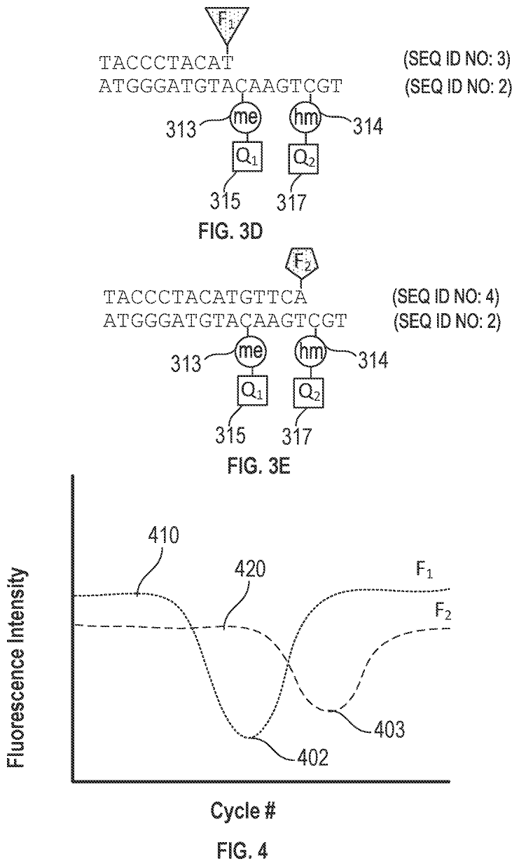

In examples where only a single type of methylated nucleotide is coupled to a quencher, such as described with reference to FIGS. 1A-1H and 2A-2C, then the identity of the methylated nucleotide may be inferred from the reduction in fluorescence caused by the presence of the quencher coupled to that methylated nucleotide. In other examples, different types of methylated nucleotides may be coupled to different quenchers than one another, and each type of methylated nucleotide may be detected and identified. For example, FIGS. 3A-3E schematically illustrate example compositions and operations in a process flow for detecting different methylated nucleotides using different quenchers respectively coupled to the different methylated nucleotides. In the nonlimiting example illustrated in FIG. 3A, a first type of methylated nucleotide 313, e.g., methylcytosine (mC) is coupled to a first type of quencher (Q₁) 315, and a second type of methylated nucleotide 314, e.g., hydroxymethylcytosine (hmC) is coupled to a second type of quencher (Q₂) 317 that is different from the first type of quencher. In some examples, the first and second methylated nucleotides 313, 314 may be selected from the group consisting of mC, hmC, fC, caC, and 6 mA. Nonlimiting examples of operations and compositions for selectively coupling different types of methylated nucleotides to different types of quenchers are provided elsewhere herein.

A polymerase (not specifically illustrated) may be used to add fluorescently labeled nucleotides to primer 316 in a manner similar to that described with reference to FIGS. 1B-1H. Different types of nucleotides may be coupled to different types of fluorophores that are selected to respectively interact with the different types of quenchers. For example, a first type of nucleotide (illustratively, T) may be coupled to a first type of fluorophore F₁ that interacts with first quencher Q₁ in a manner that decreases its fluorescence intensity as a function of proximity to Q₁, in a manner similar to that described with reference to FIGS. 1A-1H and 2A-2C. Similarly, a second type of nucleotide (illustratively, A) may be coupled to a second type of fluorophore F₂ that interacts with second quencher Q₂ in a manner that decreases its fluorescence intensity as a function of proximity to Q₂, also in a manner similar to that described with reference to FIGS. 1A-1H and 2A-2C. However, the interaction between first fluorophore F₁ and second quencher Q₂ may be significantly weaker than the interaction between first fluorophore F₁ and first quencher Q₁. Additionally, the interaction between second fluorophore F₂ and first quencher Q₁ may be significantly weaker than the interaction between second fluorophore F₂ and second quencher Q₂. Accordingly, in some examples, any reduction in fluorescence of first fluorophore F₁ substantially may be attributed to its proximity to first quencher Q₁, and any reduction in fluorescence of second fluorophore F₂ substantially may be attributed to its proximity to second quencher Q₂.

At the particular time illustrated in FIG. 3B, a polymerase (not specifically illustrated) may have added a T, labeled with first fluorophore F₁, to primer 316. At this time, first fluorophore F₁ may be sufficiently far from first quencher Q₁ as substantially not to experience a decrease in fluorescence. At the particular time illustrated in FIG. 3C, the polymerase (or another polymerase) may have added an A, labeled with second fluorophore F₂, to primer 316. At this time, second fluorophore F₂ may be sufficiently far from second quencher Q₂ as substantially not to experience a decrease in fluorescence. However, as the polymerase continues to add nucleotides, the fluorophores F₁, F₂ of some of the T and A nucleotides may come into sufficient proximity to respective quenchers Q₁, Q₂ to experience a reduction in fluorescence. For example, at the particular time illustrated in FIG. 3D, a polymerase (not specifically illustrated) may have added another T, the first fluorophore F₁ of which may be sufficiently close to first quencher Q₁ as to experience a decrease in fluorescence, as intended to be suggested by the darkened shading. At the particular time illustrated in FIG. 3E, the polymerase (or another polymerase) may have added an A, the second fluorophore F₂ of which may be sufficiently close to second quencher Q₂ as to experience a decrease in fluorescence, as intended to be suggested by the darkened shading.

The first quencher Q₁ may reduce fluorescence from first fluorophore F₁ more than it reduces fluorescence from second fluorophore F₂, such that the reduction in fluorescence of F₁ is primarily a function of distance from Q₁ and is significantly less of a function of distance from Q₂. The second quencher Q₂ may reduce fluorescence from second fluorophore F₂ more than it reduces fluorescence from first fluorophore F₂, such that the reduction in fluorescence of F₂ is primarily a function of distance from Q₂ and is significantly less of a function of distance from Q₁. In some examples, first quencher Q₁ may be selected so as to reduce at least twice as much fluorescence from first fluorophore F₁ than it does from second fluorophore F₂, or at least three times as much fluorescence from first fluorophore F₁ than it does from second fluorophore F₂, or at least four times as much fluorescence from first fluorophore F₁ than it does from second fluorophore F₂, or at least five times as much fluorescence from first fluorophore F₁ than it does from second fluorophore F₂, or at least ten times as much fluorescence from first fluorophore F₁ than it does from second fluorophore F₂. Similarly, in some examples, second quencher Q₂ may be selected so as to reduce at least twice as much fluorescence from second fluorophore F₂ than it does from first fluorophore F₁, or at least three times as much fluorescence from second fluorophore F₂ than it does from first fluorophore F₁, or at least four times as much fluorescence from second fluorophore F₂ than it does from first fluorophore F₁, or at least five times as much fluorescence from second fluorophore F₂ than it does from first fluorophore F₁, or at least ten times as much fluorescence from second fluorophore F₂ than it does from first fluorophore F₁.

For example, FIG. 4 illustrates a plot of example fluorescence intensities as a function of the number of fluorescent nucleotides added to a primer hybridized to a polynucleotide including different methylated nucleotides having different quenchers respectively coupled thereto. Trace 410 illustrated in FIG. 4 corresponds to the overall trend of fluorescence intensity from first fluorophore F₁, and trace 420 corresponds to the overall trend of fluorescence intensity from second fluorophore F₂. From FIG. 4, it may be understood that first fluorophores that are sufficiently far from first quencher Q₁ 315 (that is, fluorophores F₁ of nucleotides added in cycles that are relatively low or relatively high in number relative to the cycle in which the nucleotide is added opposite methylated nucleotide 313), may fluoresce with an intensity that is substantially unaffected by the presence of both of quenchers Q₁ and Q₂, e.g., may have a fluorescence intensity at approximately a baseline (not specifically labeled) similar to that described with reference to FIGS. 2A-2C. In comparison, first fluorophores F₁ that are sufficiently close to first quencher Q₁ may fluoresce with intensities that generally decrease as a function of distance from the quencher (that is, fluorophores added in cycles that are sufficiently close to the cycle in which the nucleotide is added opposite first methylated nucleotide 313). Similarly as discussed above with reference to FIG. 1G, the fluorescence from any first fluorophore F₁ that is added opposite methylated nucleotide 313 may be expected to be the lowest of all the nucleotides coupled to the first fluorophore, at minimum 402, because it is the closest to quencher 315.

Likewise, from FIG. 4, it may be understood that second fluorophores that are sufficiently far from second quencher Q₂ 317 (that is, fluorophores F₂ of nucleotides added in cycles that are relatively low or relatively high in number relative to the cycle in which the nucleotide is added opposite methylated nucleotide 314), may fluoresce with an intensity that is substantially unaffected by the presence of both of quenchers Q₁ and Q₂, e.g., may have a fluorescence intensity at approximately a baseline (not specifically labeled) similar to that described with reference to FIGS. 2A-2C. In comparison, second fluorophores F₂ that are sufficiently close to second quencher Q₂ may fluoresce with intensities that generally decrease as a function of distance from the quencher (that is, fluorophores added in cycles that are sufficiently close to the cycle in which the nucleotide is added opposite second methylated nucleotide 314). Similarly as discussed above with reference to FIG. 1G, the fluorescence from any second fluorophore that is added opposite methylated nucleotide 314 may be expected to be the lowest of all the nucleotides coupled to the first fluorophore, at minimum 403, because it is the closest to quencher 317.

Note that in the nonlimiting example shown in FIG. 4, second quencher Q₂ 317 substantially does not interact with first fluorophore F₁, and first quencher Q₁ 315 substantially does not interact with second fluorophore F₂. Accordingly, trace 410 substantially does not include any features corresponding to a reduction in fluorescence of first fluorophore F₁ by second quencher Q₂ 317, and trace 420 substantially does not include any features corresponding to a reduction in fluorescence of second fluorophore F₂ by first quencher Q₁ 315. In some examples, one or both of the first and second quenchers interact with both of the first and second fluorophores, or examples in which one or both of the first and second fluorophores interact with both of the first and second quenchers. Accordingly, in some examples, trace 410 may include a first minimum 402 corresponding to interaction between the first fluorophore and the first quencher, and a second minimum (not specifically illustrated, but located at the same cycle number as minimum 403) corresponding to interaction between the first fluorophore and the second quencher. Because the interaction between the first fluorophore and the first quencher is stronger than the interaction between the first fluorophore and the second quencher, the first minimum of trace 410 may be deeper than the second minimum of trace 410. Illustratively, in nonlimiting examples in which the first quencher reduces fluorescence of the first fluorophore at least twice as much as the second quencher reduces fluorescence of the first fluorophore, then the first minimum 402 of trace 410 may be expected to be at least twice as deep as the second minimum of trace 410. Additionally, or alternatively, in some examples, trace 420 may include a first minimum (not specifically illustrated, but located at the same cycle number as minimum 402) corresponding to interaction between the second fluorophore and the first quencher, and a second minimum 403 corresponding interaction between the second fluorophore and the second quencher. Because the interaction between the second fluorophore and the second quencher is stronger than the interaction between the second fluorophore and the first quencher, the second minimum 403 of trace 420 may be deeper than the first minimum of trace 420. Illustratively, in nonlimiting examples in which the second quencher reduces fluorescence of the second fluorophore at least twice as much as the first quencher reduces fluorescence of the second fluorophore, then the second minimum 403 of trace 420 may be expected to be at least twice as deep as the first minimum of trace 420.

The respective locations of the first and second methylated nucleotides 313, 314 within the polynucleotide being analyzed may be determined in a manner similar to that described with reference to FIGS. 2B-2C. For example, in a manner similar to that described with reference to FIG. 2B, the fluorescence intensity measured during a given cycle may be discontinuous with fluorescence intensities measured during the previous and subsequent cycles. Additionally, in a manner similar to that described with reference to FIG. 2C, the fluorescence intensities measured during different cycles may have different magnitudes because the fluorophores have different intrinsic fluorescent intensities than one another. For examples such as described with reference to FIGS. 3A-3E and 4, appropriate processing circuitry may be used to determine the respective locations of minima within the fluorescence intensities from respective fluorophores F₁, F₂, and thus to determine the respective locations of the methylated nucleotides 313, 314 to which the first and second quenchers 315, 317 respectively are coupled. For example, similarly as described before a base calling algorithm may be generated by using the present operations to sequence known DNA sequences including methylated nucleotides respectively coupled to different quenchers (e.g., within any suitable number of one or more CpG sites). The measured reductions in fluorescence caused by the different quenchers may be recorded, and bioinformatically used to create a base calling algorithm that may be used to detect similar reductions in fluorescence and correlate such reductions to the respective presence of methylated nucleotides coupled to different quenchers.

In examples such as described with reference to FIGS. 3A-3E and 4, different types of methylated nucleotides may be coupled to a respective, different type of quencher. As such, the identity of a methylated nucleotide may be inferred from the reduction in fluorescence caused by the presence of the particular type of quencher coupled to that methylated nucleotide. For example, because fluorescence from the first fluorophore F₁ selectively is inhibited by first quencher Q₁, and first quencher Q₁ selectively is coupled to methylcytosine, minimum 402 in trace 410 may be used to determine that methylcytosine was present at the location corresponding to minimum 402. Similarly, because fluorescence from the second fluorophore F₂ selectively is inhibited by second quencher Q₂, and second quencher Q₂ selectively is coupled to hydroxymethylcytosine, minimum 403 in trace 420 may be used to determine that hydroxymethylcytosine was present at the location corresponding to minimum 403.

Quenchers for use in selectively reducing fluorescence from respective fluorophores may be selected using the teachings provided herein. For example, a wide variety of fluorophores are used in commercially available sequencing by synthesis equipment. Quenchers may be selected based on spectral overlap between the fluorophores and the quenchers, and desire behavior. For example, some quenchers may have a relatively narrow absorption spectrum and may be used to quench only a certain type or certain types of fluorophores that are used for sequencing by synthesis, while other quenchers may have a relatively broad absorption spectrum and may be used to quench some or all types of fluorophores that are used for sequencing by synthesis.

From the foregoing, it should be understood that any suitable number of different types of methylated nucleotides may be coupled to respective types of quenchers, and the respective reductions in fluorescence caused by such quenchers may be used to determine the presence and identify of the nucleotide. Illustratively, the methylated nucleotide(s) may include any suitable combination of one or more of 5-methylcytosine (5-mC), 5-hydroxymethylcytosine (5-hmC), 5-formylcytosine (5-fC), 5-carboxylcytosine (5-caC), or 6-methyladenine (6-mA). In some examples, only one type of methylated nucleotide is coupled to a quencher. In some examples, two different types of methylated nucleotides are coupled to respective, different quenchers. In some examples, three different types of methylated nucleotides are coupled to respective, different quenchers. In some examples, four different types of methylated nucleotides are coupled to respective, different quenchers. Five or more different types of methylated nucleotides also can be coupled to respective, different quenchers.

The polynucleotide being sequenced, and within which methylated nucleotides are identified, may be sequenced in any suitable manner. In some examples, the polynucleotide being sequenced is coupled to a substrate. For example, the polynucleotide may be located within a cluster of polynucleotide amplicons coupled to the substrate. In some examples, the polynucleotide amplicons of the cluster also respectively include first methylated nucleotides. The quencher may be coupled to the methylated nucleotides of the respective amplicons in a manner such as provided herein, e.g., with reference to FIG. 1B for a single quencher, or with reference to FIG. 3A for multiple types of quenchers. Fluorescently labeled nucleotides may be added to primers respectively hybridized to the amplicons, e.g., in a manner such as described with reference to FIGS. 1D-1H or 3B-3E. The quencher may be used to reduce fluorescence from at least one of the added, fluorescently labeled nucleotides, e.g., in a manner such as described with reference to FIGS. 1E-1H or 3D-3E. The reduced fluorescence caused by the quencher may be used to detect the methylated nucleotide in the respective amplicons, e.g., in a manner such as described with reference to FIGS. 2A-2C and 4.

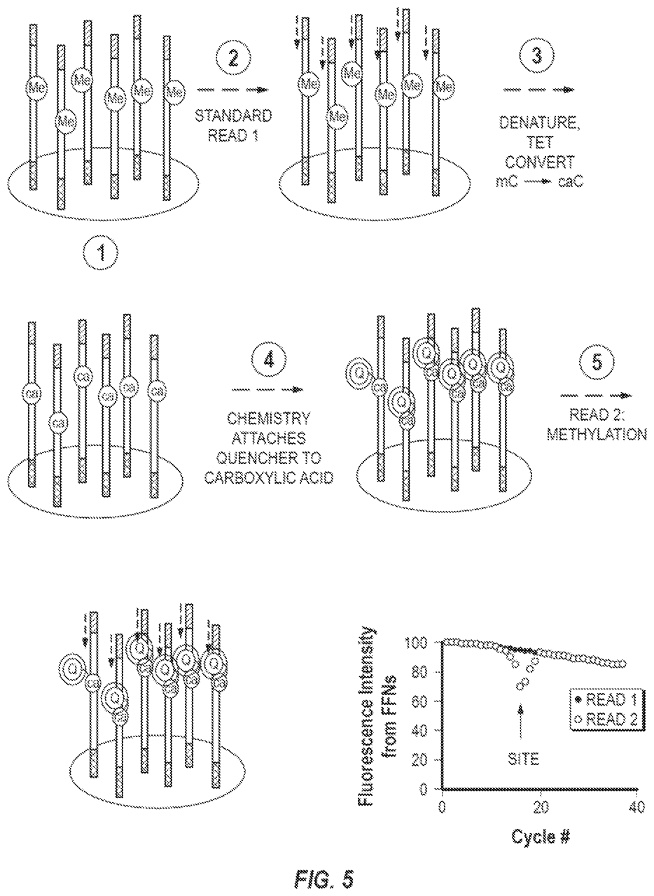

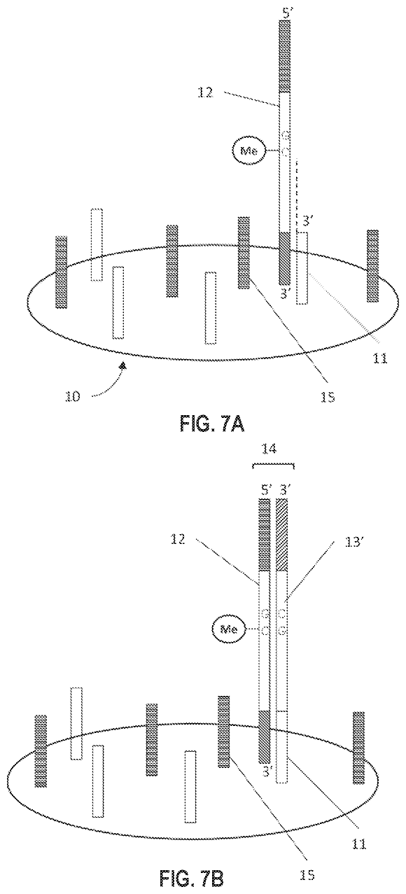

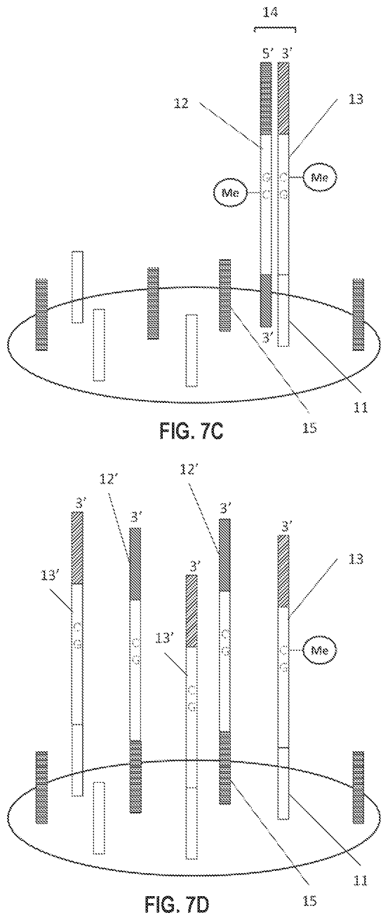

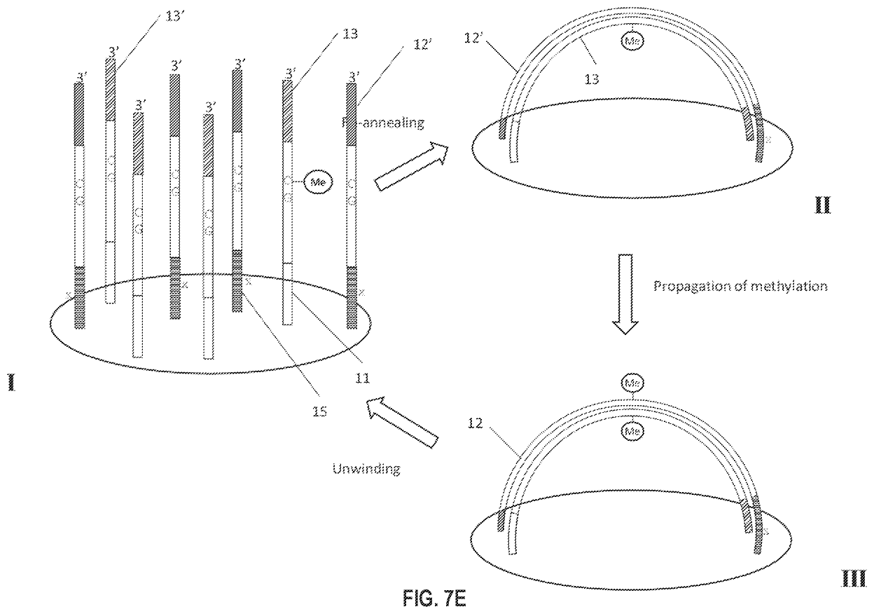

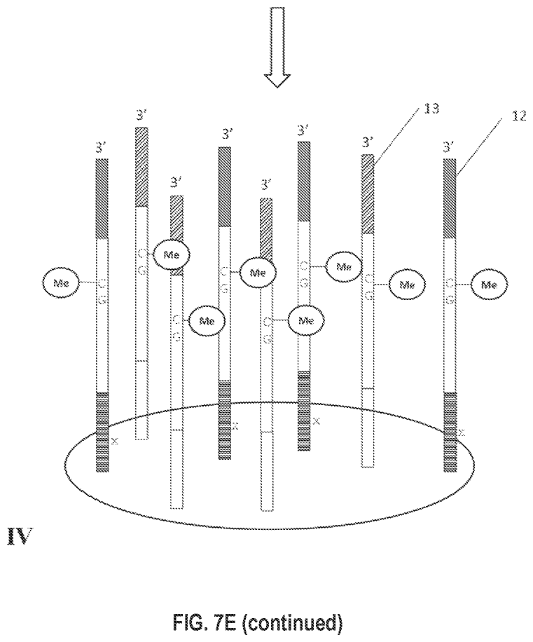

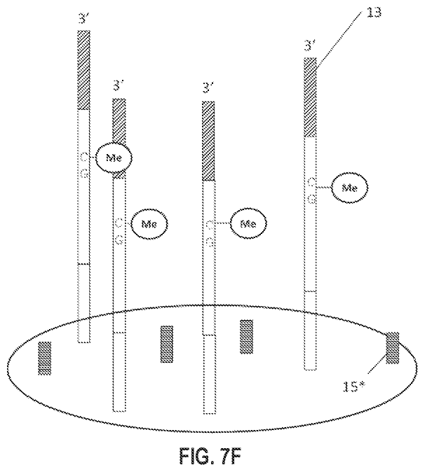

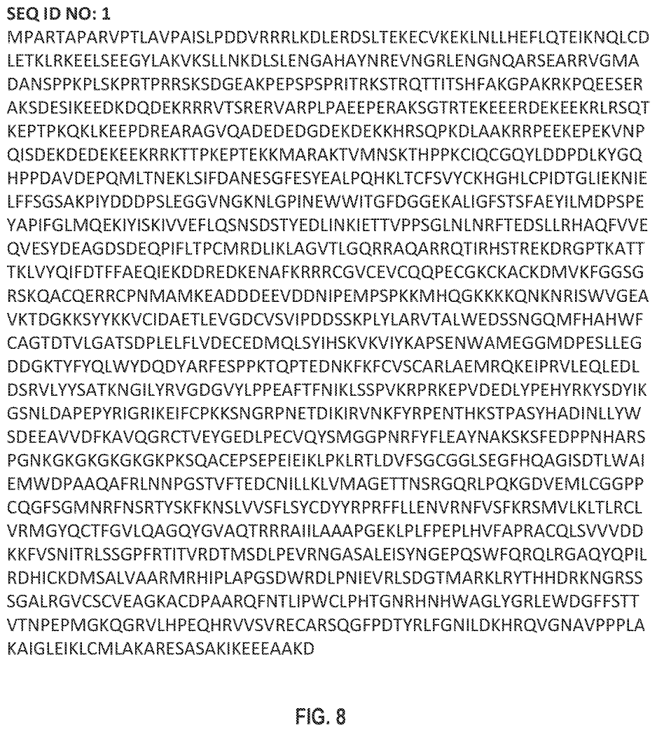

FIG. 5 schematically illustrates example compositions and operations in a process flow for detecting methylated nucleotides within a cluster of amplicons. At operation 1 illustrated in FIG. 5, a cluster of amplicons of a polynucleotide is generated, wherein at least some of the amplicons include the methylated nucleotide. In some examples, substantially all of the amplicons include the methylated nucleotide. Illustratively, DNMT1 enzyme may be used to maintain symmetric methylation on the flow cell during clustering, that is, may be used to methylate cytosine at locations that correspond to methylcytosine in the original polynucleotide. For simplicity, the amplicons are illustrated as including a single methylated nucleotide, e.g., a single methylcytosine. However, it will be appreciated that the amplicons may include a plurality of methylated nucleotides of the same type as one another or of different types. FIG. 8 shows an example sequence of the human template-dependent DNA (cytosine-5)-methyltransferase 1 (DNMT1, SEQ ID NO:1). DNMT1 preferentially identifies hemi-methylated CpG dinucleotide sites. A hemi-methylated CpG dinucleotide, also referred to as a hemi-methylated site, describes a situation where a cytosine of a CpG dinucleotide is methylated on one strand but the cytosine of the complementary CpG dinucleotide on the other strand is not methylated. DNMT1 methylates the cytosine of the complementary CpG dinucleotide, converting the hemi-methylated site to CpG dinucleotides on both strands. However, wild-type DNMT1 is unable to survive the high temperatures encountered during PCR cycling. For this reason, DNMT1-based methyl-CpG amplification can be used either with an engineered thermostable DNMT1 or the addition of fresh DNMT1 following each PCR cycle. DNA methyltransferase enzymes are commercially available, for example from Sigma Aldrich™ (catalog no. SRP0126) and from Active Motif™ (catalog no. 31404). Further details about how to maintain symmetric methylation on the flow cell during clustering are provided below with reference to FIGS. 7A-7F.

At operation 2 illustrated in FIG. 5, a standard sequencing-by-synthesis read 1 procedure is performed to determine the sequence of the amplicons. For example, primers may be hybridized to the amplicons, and a polymerase used to add fluorescently labeled nucleotides (FFNs) to the primer. The sequence of the polynucleotide is complementary to the sequence of fluorescently labeled nucleotides that are added.

At operation 3 illustrated in FIG. 5, the extended primer is removed using denaturing. A quencher then may be coupled to the methylated nucleotides of the amplicons, e.g., using any suitable combination of operations provided herein. In the nonlimiting example illustrated in FIG. 5, any 5-mC in the amplicons is oxidized to 5-caC. Such oxidation optionally may be performed using a ten-eleven translocation (TET) enzyme, or using a chemical reagent, e.g., in a manner such as described in greater detail below. At operation 4 illustrated in FIG. 5, the 5-carboxyl group of the 5-caC is coupled to the quencher (Q), e.g., in a manner such as described in greater detail below. For simplicity, the amplicons are illustrated as having a single methylated nucleotide coupled to a single quencher, it will be appreciated that the amplicons may include several methylated nucleotides which respectively may be coupled to quenchers. Additionally, in some examples, the amplicons may include different types of nucleotides that optionally may be coupled to different types of quenchers in a manner such as described with reference to FIGS. 3A-3E.

At operation 5 illustrated in FIG. 5, a sequencing-by-synthesis read 2 procedure is performed to determine the sequence of the amplicons. For example, another set of primers may be hybridized to the amplicons, and a polymerase used to add FFNs to the primer. Similarly as for read 1, the sequence of the polynucleotide is complementary to the sequence of fluorescently labeled nucleotides that are added. However, in read 2, the quenchers inhibit fluorescence from fluorophores that are added at locations that are sufficiently close to the quenchers. Accordingly, as illustrated in the plot shown in FIG. 5, the fluorescence intensity during read 2 may include a local minimum corresponding to the location of the methylated nucleotide, e.g., in a manner such as described with reference to FIGS. 2A-2C or 4. The methylated nucleotide may be identified using the wavelength of fluorescence from the complementary, fluorescently labeled nucleotide added during read 1 and/or read 2. Note that if there was sufficient fluorescence during read 2 to sequence the amplicons with desired accuracy and also to identify the methylated nucleotides, then read 1 may not be necessary and optionally may be omitted.

Nonlimiting, purely illustrative examples of reaction schemes that selectively may be used to couple quenchers to different types of methylated nucleotides now will be provided.

In some examples, the quencher is coupled to 5-caC, and the 5-carboxyl group of the 5-caC may be reacted with a molecule to form a product. The 5-caC may be naturally occurring in the polynucleotide, or may be generated by oxidizing 5-mC, 5-hmC, or 5-fC in the polynucleotide to 5-caC. The oxidation may be performed using any suitable combination of chemical and/or enzymatic reagents. In some examples using an enzymatic reagent, a ten-eleven translocation (TET) dioxygenase is used to oxidize the 5-mC, 5-hmC, or 5-fC to 5-caC. In some nonlimiting examples using one or more chemical reagents, 5-mC may be oxidized to 5-caC using menadione, ultraviolet (UV) radiation at 365 nm, under oxygen, followed by 2,2,6,6-tetramethyl-1-piperidinyloxy free radical (TEMPO)/bis(acetoxyiodobenzene) (BAIB) in a manner such as described in Kore et al., “Concise synthesis of 5-methyl, 5-formyl, and 5-carboxy analogues of 2′-deoxycytidine-5′-triphosphate,” Tetrahedron letters 54 (39): 5325-5327 (2013), the entire contents of which are incorporated by reference herein. In other nonlimiting examples using one or more chemical reagents, 5-hmC or 5-fC may be oxidized to 5-caC using TEMPO/BAIB in a manner such as described in Sun et al., “Efficient synthesis of 5-hydroxymethyl-, 5-formyl-, and 5-carboxyl-2′-deoxycytidine and their triphosphates,” RSC Advances 4(68): 36036-36039 (2014), the entire contents of which are incorporated by reference herein. In still other nonlimiting examples using one or more chemical reagents, an iron (IV)-oxo complex is used to oxidize 5-mC to 5-caC in a manner such as described in Schmidl et al., “Biomimetic iron complex achieves TET enzyme reactivity,” Angewandte Chemie Int'l Ed. 60 (39): 21457-21463 (2021), the entire contents of which are incorporated by reference herein.

The 5-carboxyl group of the 5-caC may be reacted with the molecule in any suitable manner. For example, the reaction may include activating the 5-carboxyl group of the 5-caC before reacting the 5-carboxyl group with the molecule. Illustratively, the 5-carboxyl group of the 5-caC may be activated using 4-(4,6-dimethoxy-1,3,5-triazin-2-yl)-4-methyl-morpholinium chloride (DMTMM) or 1-ethyl-3-(3′-(dimethylamino) propyl) carbodiimide (EDC). The molecule with which the activated carboxylic acid is reacted may include a nucleophile. In some examples, the product of reaction between the carboxyl group of the 5-caC and the molecule includes the quencher, while in other examples the quencher is subsequently coupled to the product. A nonlimiting example of a reaction scheme in which EDC is used to activate the 5-carboxyl group of 5-caC and a nucleophile (Nu) then added that includes the quencher, or a functional group to which a quencher subsequently may be coupled (such as biotin or N₃), is shown below:

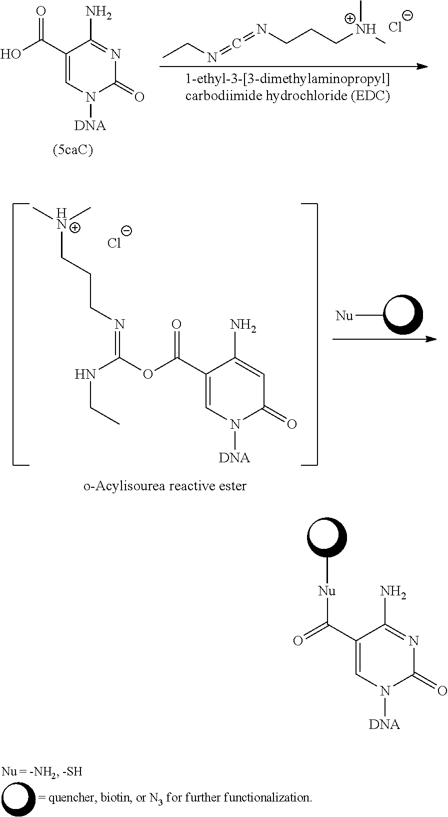

For further details regarding using EDC to activate the 5-carboxyl group of 5-caC into a reactive ester that can react with a nucleophile, see Lu et al., “Chemical modification-assisted bisulfite sequencing (CAB-seq) for 5-carboxylcytosine detection in DNA,” J. Am. Chem. Soc. 135(25): 9315-9317 (2013), the entire contents of which are incorporated by reference herein.

A nonlimiting example of a reaction scheme in which DMTMM is used to activate the 5-carboxyl group of 5-caC and a nucleophile (Nu) then added that includes the quencher, or a functional group to which a quencher subsequently may be coupled (such as biotin or N₃), is shown below:

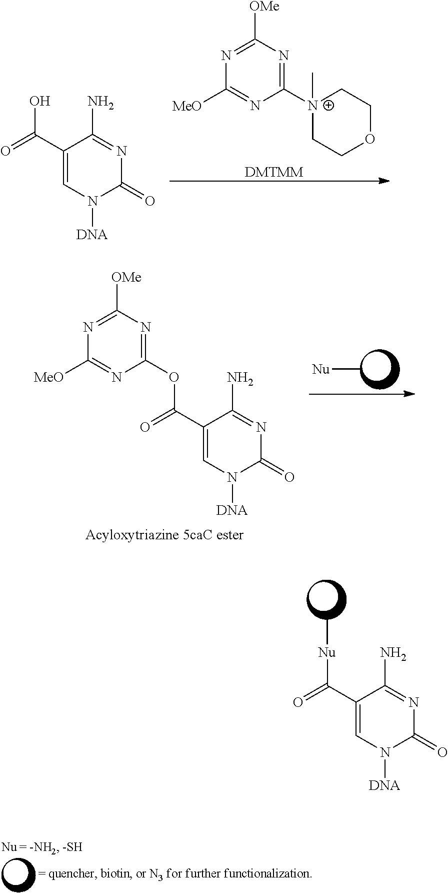

For further details regarding using DMTMM to activate the 5-carboxyl group of 5-caC into a reactive ester that can react with a nucleophile, see Xie et al., “Selective chemical labeling and sequencing of 5-carboxylcytosine in DNA at single-base resolution,” Analytical Chemistry 92(18): 12710-12715 (2020), the entire contents of which are incorporated by reference herein.

It will be appreciated that still other suitable conjugation techniques may be used to couple a molecule to the 5-carboxyl group of 5-caC. Illustratively, the molecule may include an azirine. The molecule also may include the quencher or a functional group to which a quencher subsequently may be coupled (such as biotin or N₃). A nonlimiting example of a reaction scheme in which the azirine may react with the 5-carboxyl group of 5-caC, to couple the molecule to the 5-caC, is shown below:

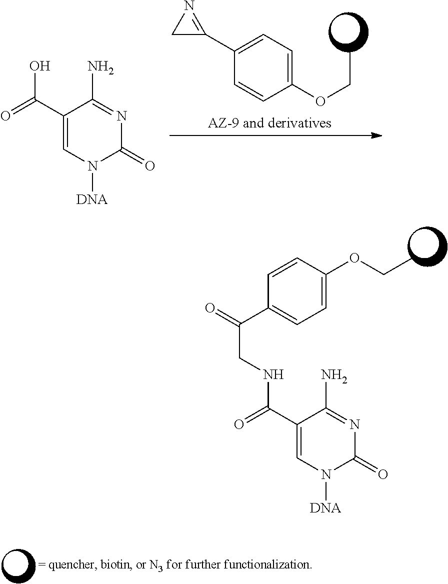

In the foregoing scheme, 4-(2H-azyrin-3-yl)-phenol (AZ-9) and 4-(2H-azyrin-3-yl)-phenyl derivatives are intended to encompass different functionalizations that are compatible with this chemistry, e.g., the identities of the black sphere illustrated above (e.g., quencher, biotin, or N₃). 4-(2H-azyrin-3-yl)-phenol alternatively may be functionalized with an alkyne, but it should be appreciated that any substituted 3-phenyl-2H-azirine may be used, wherein the substitution includes a quencher or may be coupled to a quencher. For further details regarding using an azirine (such as 4-(2H-azyrin-3-yl)-phenol) to couple a molecule to a carboxyl group in a biologically compatible manner, see Ma et al., “2H-Azirine-based reagents for chemoselective bioconjugation at carboxyl residues inside live cells,” J. Am. Chem. Soc. 142(13): 6051-6059 (2020), the entire contents of which are incorporated by reference herein.

Another example of a methylated nucleotide that can be coupled to a quencher is 5-mC. In examples such as provided above, 5-mC may be oxidized enzymatically or chemically to 5-caC and then reacted with a molecule that includes, or may be coupled to, a quencher. However, other reaction schemes may be used to couple the molecule to 5-mC without the need for oxidizing the 5-mC to 5-caC, e.g., such as now will be described.

In some examples, the quencher may be coupled to 5-mC using operations that include reacting the 5-methyl group of the 5-mC with a first molecule to form a product; and reacting the product with a second molecule to couple the first quencher to the first product. Illustratively, reacting the methyl group of the 5-mC with the first molecule may include using CMD1 to couple the first molecule (e.g., a cis-diol) to the 5-methyl group. CMD1 is a homolog of TET, and uses 5-mC and vitamin C as substrates. For further details regarding CMD1, see Li et al., “Molecular mechanism for vitamin C-derived C5-glyceryl-methylcytosine DNA modification catalyzed by algal TET homologue CMD1,” Nature Communications 12: article number 744 (2021), the entire contents of which are incorporated by reference herein. As provided herein, the product of such reaction may include a diol to which a suitable second molecule may be coupled. In the nonlimiting example shown below, the second molecule may include a boronate (—B(OH)₂) that coordinates to the diol:

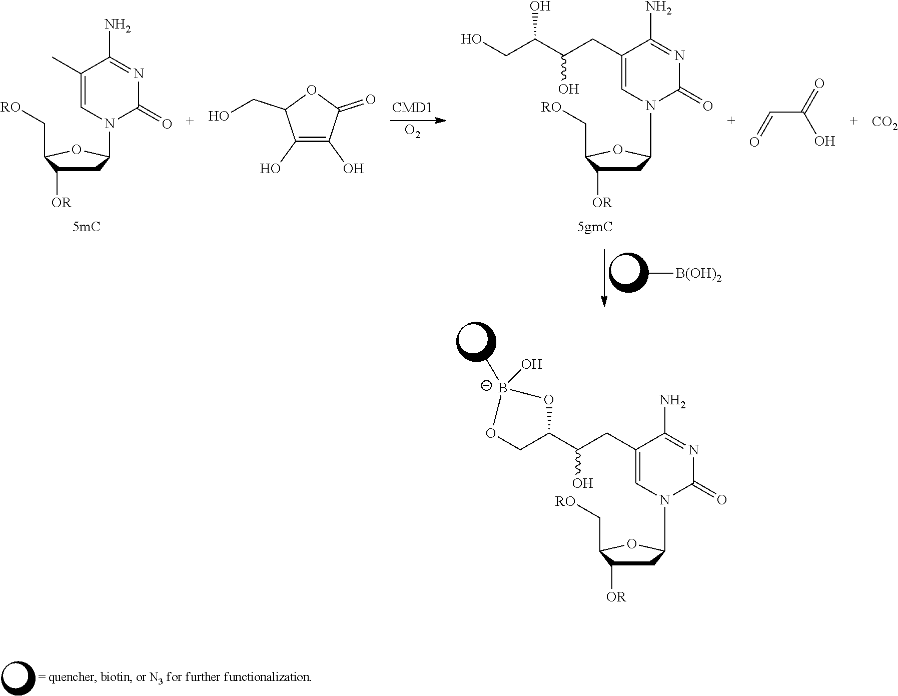

Boronates are selective for diols, work in mild conditions (e.g., a pH of about 7-8), and can be removed if desired because their reaction with the diol is reversible. In some examples, the boronate may be a phenyl boric acid derivative. For further details regarding coordinating boronates (such as phenyl boric acid derivatives) to diols, see Brooks et al., “Structure-reactivity relationships in boronic acid-diol complexation,” ACS Omega 3: 17863-17870 (2018), the entire contents of which are incorporated by reference herein.

In other examples, the quencher may be coupled to the 5-mC using operations that include oxidizing the 5-mC to 5-hmC; reacting the 5-hydroxymethyl group of the 5-hmC with a first molecule to form a first product; and reacting the first product with a second molecule to couple the first quencher to the first product. The 5-mC may be oxidized to 5-hmC using any suitable combination of enzymatic and/or chemical reagents. In one nonlimiting example, a TET dioxygenase is used to oxidize the 5-mC to the 5-hmC, illustratively ccTET, which is a TET homolog from a fungus. For further details regarding ccTET, see Zhang et al., “A TET homologue protein from Coprinopsis cinerea (CcTET) that biochemically converts 5-methylcytosine to 5-hydroxymethylcytosine, 5-formylcytosine, and 5-carboxylcytosine,” J. Am. Chem. Soc. 136 (13): 4801-4804 (2014), the entire contents of which are incorporated by reference herein. For other nonlimiting examples of enzymes that may be used to oxidize 5-mC to 5-hmC, see Liu et al., “Mutations along a TET2 active site scaffold stall oxidation at 5-hydroxymethylcytosine,” Nat. Chem. Biol. 13(2): 181-187 (2017), the entire contents of which are incorporated by reference herein.

As provided herein, the 5-hydroxymethyl group of the 5-hmC then may be reacted with the first molecule. In some examples, the reaction of the hydroxymethyl group of the 5-hmC with the first molecule may include using Mha.I to couple the first molecule to the hydroxymethyl group. Mha.I is an evolved methylase (Msss.I) that may be used to functionalize 5-hmC. More specifically, Mha.I is a methyltransferase that, when lacking the SAM cofactor, can transfer a thiol onto 5-hmC. This is achieved by nucleophilic attack of a cysteine onto the C6 of 5-hmC, which triggers loss of water from C5 and C5′. Then, the thiol nucleophile attacks the C5-C5′ alkene, and liberates the covalent enzyme intermediate. For further details regarding using Mha.I to derivative 5-hmC with a thiol, see Liutkeviciute et al., “Methyltransferase-directed derivatization of 5-hydroxymethylcytosine in DNA,” Angew. Chem. Int. Ed. Engl. 50(9): 2090-2093 (2011), the entire contents of which are incorporated by reference herein. In the nonlimiting scheme illustrated below, MhA.I is used to couple an aminothiol to the 5-hmC. The aminothiol then is reacted with any suitable second molecule, such as an N-hydroxysuccinimide (NHS) ester, an isocyanate, or an isothiocyanate:

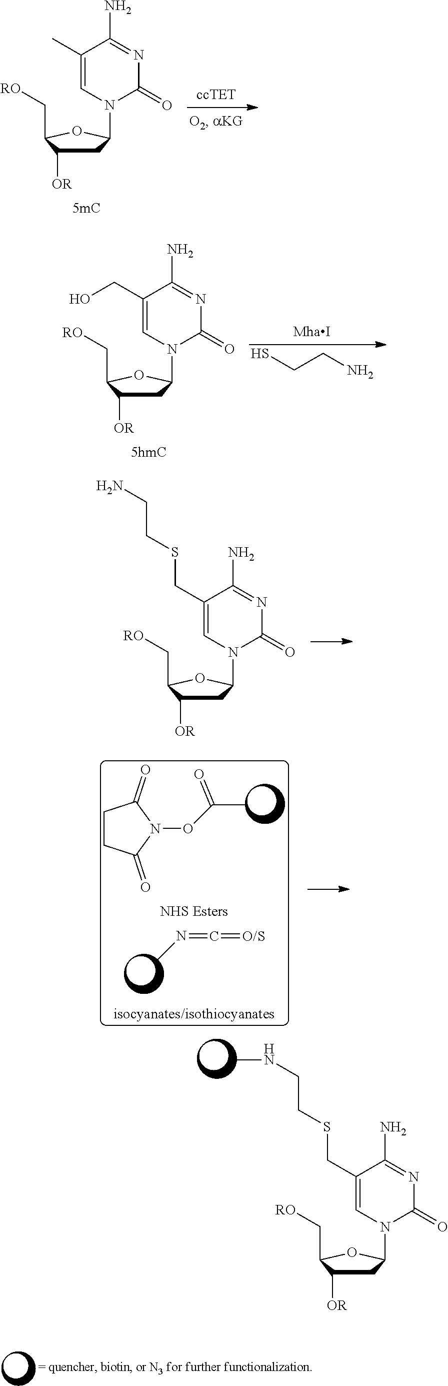

The second molecule may include a quencher, or may include a functional group (such as biotin or N₃) to which a quencher subsequently may be coupled.

Yet another example of a methylated nucleotide that can be coupled to a quencher is 5-hmC. In examples such as provided above, 5-hmC may be oxidized enzymatically or chemically to 5-caC and then reacted with a molecule that includes, or may be coupled to, a quencher. However, other reaction schemes may be used to couple the molecule to 5-hmC without the need for oxidizing the 5-hmC to 5-caC. For example, the quencher may be coupled to the 5-hmC using operations that include reacting the 5-hydroxymethyl group of the 5-hmC with a first molecule to form a first product; and reacting the first product with a second molecule to couple the first quencher to the first product. In one non-limiting example, Mha.I may be used to couple the first molecule (e.g., aminothiol) to the hydroxymethyl group of 5-hmC in a similar manner as described above with reference to 5-mC. The first product then may be coupled to a second molecule (e.g., NHS ester, isocyanate, or isothiocyanate) in a similar manner as described above with reference to 5-mC. Accordingly, it should be appreciated that a similar reaction scheme may be used to couple a quencher to naturally occurring 5-mC that is oxidized to 5-hmC (e.g., using ccTET), or may be used to couple a quencher to naturally occurring 5-hmC.

Yet another example of a methylated nucleotide that can be coupled to a quencher is 6-mA. For example, the quencher may be coupled to 6-mA using an FTO-assisted chemical labeling method termed m⁶A-SEAL. For further details regarding m6A-SEAL, see Wang et al., “Antibody-free enzyme-assisted chemical approach for detection of N6-methyladenosine,” Nature Chemical Biology 16:896-903 (2020), the entire contents of which are incorporated by reference herein. In some examples, the quencher may be attached with the same strategy used in Wang (but by replacing a quencher with the biotin) or with any of the commercially available thiol specific chemistries, for example such as described in Ochtrop et al., “Recent advances of thiol-selective bioconjugation reactions,” Current Opinion in Chemical Biology 58:28-36 (2020), the entire contents of which are incorporated by reference herein.

Furthermore, it should be appreciated that the reaction schemes provided herein respectively are selective for a particular type of methylated nucleotide, and thus may be expected to couple a quencher selectively to that type of methylated nucleotide. For example, carbonyl activation and nucleophilic attack may be expected to substantially react with the 5-carboxyl group of 5-caC, and substantially may not be expected to react with 5-mC, 5-hmC, or 5-fC. Similarly, azirines may be expected to substantially react with the 5-carboxyl group of 5-caC, and substantially may not be expected to react with 5-mC, 5-hmC, or 5-fC. Likewise, CMD1 may be expected to react selectively with 5-mC and substantially may not be expected to react with 5-hmC, 5-fC, or 5-caC. Similarly, ccTET may be expected to react selectively with 5-mC and substantially may not be expected to react with 5-hmC, 5-fC, or 5-caC. Likewise, Mha.I may be expected to react selectively with 5-hmC and substantially may not be expected to react with 5-mC, 5-fC, or 5-caC. Accordingly, in some examples provided herein, different reaction schemes may be used to selectively couple different types of quenchers to different types of methylated nucleotides.

It will be appreciated that the reaction product may include any suitable functional group that may be coupled to a quencher. For example, the functional group may include a SNAP protein and the quencher may include O-benzylguanine. Or, for example, the functional group may include a CLIP protein and the quencher may include an O-benzylcytosine. Or, for example, the functional group may include Spy Tag and the quencher may include SpyCatcher. Or, for example, the functional group may include SpyCatcher and the quencher may include SpyTag. Or, for example, the functional group may include biotin and the quencher may include streptavidin. Or, for example, the functional group may include streptavidin and the quencher may include biotin. Or, for example, the functional group may include NTA and the quencher may include His-Tag. Or, for example, the functional group may include His-Tag and the quencher may include NTA. Or, for example, the functional group may include N₃ and the quencher may include an alkyne. Or, for example, the functional group may include an alkyne and the functional group may include N₃. Other example binding partners that may be used include, but are not limited to: SnoopTagJr and Dogtag the coupling of which may be catalyzed using SnoopLigase in a manner such as described in Buldun et al., “SnoopLigase catalyzes peptide-peptide locking and enables solid-phase conjugate isolation,” J. Am. Chem. Soc. 140(8): 3008-3018 (2018), the entire contents of which are incorporated by reference herein; suitably positioned glycine residues and nucleophiles the coupling of which may be mediated using a sortase in a manner such as described in Guimaraes et al., “Site-specific C-terminal and internal loop labeling of proteins using sortase-mediated reactions,” Nature Protocols 8:1787-1799 (2013), the entire contents of which are incorporated by reference herein; Strep-tag II and streptavidin in a manner such as described in Schmidt et al., “The Strep-tag system for one-step purification and high-affinity detection or capturing of proteins,” Nature Protocols 2:1528-1535 (2007), the entire contents of which are incorporated by reference herein; and engineered split enteins, such as NpuGEP and CfaGEP, such as described in Stevens et al., “A promiscuous split entein with expanded protein engineering applications,” PNAS 114(32): 8538-8543 (2017), the entire contents of which are incorporated by reference herein. Still other suitable combinations of functional groups readily may be envisioned.

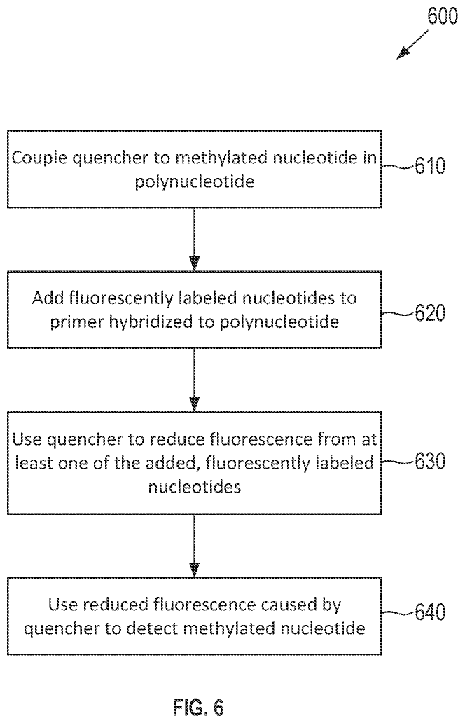

From the teachings herein, it will be understood that any suitable quencher(s) may be coupled to any suitable methylated nucleotide(s), and respectively used to detect the methylated nucleotide(s). For example, FIG. 6 illustrates a flow of operations in an example method for detecting a methylated nucleotide using a quencher coupled to the methylated nucleotide. Method 600 illustrated in FIG. 6 may include coupling a quencher to a methylated nucleotide in a polynucleotide (operation 610), for example in a manner such as described with reference to FIG. 1B, FIG. 3A, or operation 4 of FIG. 5. Nonlimiting examples of quenchers, methylated nucleotides, and operations for coupling quenchers to methylated nucleotides, are provided elsewhere herein. Method 600 also may include adding fluorescently labeled nucleotides to a primer hybridized to the polynucleotide (operation 620), for example in a manner such as described with reference to FIGS. 1D-1H, FIGS. 3B-3E, or operation 5 of FIG. 5. Method 600 also may include using the quencher to reduce fluorescence from at least one of the added, fluorescently labeled nucleotides (operation 630), for example in a manner such as described with reference to FIGS. 1E-1H, FIGS. 3D-3E, or operation 5 of FIG. 5. Method 600 also may include using the reduced fluorescence caused by the quencher to detect the methylated nucleotide (operation 640), for example in a manner such as described with reference to FIGS. 2A-2C, 4, or the plot in FIG. 5.

In some examples, the polynucleotide that is sequenced using method 600 includes a second methylated nucleotide which it is also desired to detect. The method may be modified to include coupling a second quencher to the second methylated nucleotide, for example using a different set of reactions than was used to couple the quencher to the other nucleotide. The method may include using the second quencher to reduce fluorescence from at least one of the added, fluorescently labeled nucleotides. Such operation may be performed concurrently with operation 630 described above. The method also may include using the reduced fluorescence caused by the second quencher to detect the second methylated nucleotide. Such operation may be performed concurrently with operation 640 above.

As noted above with reference to FIG. 5, a cluster of amplicons of a polynucleotide may be generated, wherein at least some of the amplicons include the methylated nucleotide. In some examples, substantially all of the amplicons include the methylated nucleotide. A nonlimiting example now will be provided of a manner in which a plurality of amplicons may be generated of a polynucleotide that includes methylated nucleotides, in which the amplicons also include the methylated nucleotides—that is, in which methylation is substantially preserved during amplification of the target polynucleotide.

FIGS. 7A-7F schematically illustrate example compositions and operations in a process flow for producing clonal clusters that preserve the CpG methylation state of a target polynucleotide. For simplicity, only one amplification site 10 of an array and a limited number of amplicons of a target polynucleotide are shown. The figures use the following convention when numbering polynucleotides: strands including a methylated cytosine are numbered (e.g., strand 13 of FIG. 7C); strands including a non-methylated cytosine are also numbered but the number is modified with the prime symbol “‘” (e.g., strand 13’ of FIG. 7B).

In some examples, the method is performed using an array that includes a plurality of amplification sites 10 such as illustrated in FIG. 7A. Each amplification site 10 includes a plurality of orthogonal capture primers 11, 15 attached to the amplification site. As shown in FIG. 7A, individual amplification sites can include a single-stranded (ss) polynucleotide 12 which is attached to the amplification site surface by hybridization between an adapter of the polynucleotide (installed during library preparation) and one of the capture primers. For example, as shown in FIG. 7A, polynucleotide 12 is shown annealed to a capture primer 11. Polynucleotide 12 may be referred to as a template, a target polynucleotide, and/or a seed polynucleotide, and in some examples may be a member of a sequencing library that has been exposed to conditions that preserve an epigenetic marker present, such as the methylation state of one or more nucleotides in the polynucleotide. In some examples, the sequencing library has been produced using methods that do not include amplification. For example, in FIG. 7A the polynucleotide 12 is shown with a CpG dinucleotide where the C is methylated. The skilled person will recognize that a single-strand polynucleotide could include any number of methylated nucleotides, including multiple distinct CpG dinucleotides where the C is methylated.

The method further includes extending the 3′ end of the capture primer 11 with a polymerase (not specifically shown), using the methylated single-strand polynucleotide 12 as template strand, to produce a complementary strand which is unmethylated. For example, as shown in FIG. 7B, such extension generates a double-stranded (ds) polynucleotide 14, where one strand includes polynucleotide 12, and the other strand includes the capture primer 11 and the newly synthesized amplicon 13′, which is the unmethylated complement of the template strand 12. The complement of methylated CpG dinucleotide of strand 12 is present on the complementary strand 13′, but the methylation status is not preserved, resulting in a hemi-methylated state for that dinucleotide present on the double-stranded polynucleotide 14.

The method includes exposing the array to conditions that transfer the hemi-methylated CpG dinucleotides of the original single-strand polynucleotide 12 to the complementary strand 13′. As used herein, when a methylation status is “transferred” from a hemimethylated CpG dinucleotide to the complementary strand, the resulting product is a CpG dinucleotide wherein both Cs are methylated. In some examples, the conditions include exposing the amplification sites to an enzyme, such as DNMT1, described elsewhere herein and for which an example sequence is shown in FIG. 8. For example, as shown in FIG. 7C, the enzyme transfers the methylation state of the methylated CpG dinucleotide of the template strand 12 to complementary strand 13′, to generate the now methylated complementary strand 13, converting the hemi-methylated site to methylated CpG dinucleotides on both strands 12, 13. It should be apparent to one skilled in the art that such a treatment would not result in methylation at CpG sites that were not originally hemimethylated, e.g., a nonmethylated CpG site would remain unmethylated after treatment. Note that strand 13 is covalently coupled to the amplification site 10 via capture primer 11, while strand 12 is noncovalently coupled to the amplification site 10 and may be removed by dehybridization.

Following transfer of the methylation status of the methylated CpG dinucleotides to the complementary strand 13 at amplification sites, the double-stranded polynucleotide 14 at each amplification site 10 is amplified to include a clonal population of immobilized polynucleotides. As illustrated in FIG. 7D, the clonal population includes a first sub-population of single-strand polynucleotides 13′ having the same nucleotide sequence as strand 13 generated in a manner such as described with reference to FIGS. 7A-7C, but the CpG dinucleotides in strands 13′ are not methylated. Strand 13 retains its methylated CpG dinucleotides produced using operations including the above-described transfer using, for example, the enzyme DNMT1. The clonal population at amplification site 10 also includes a second sub-population of single-stranded polynucleotides 12′ that include the nucleotide sequence of the template strand 12, but do not contain any methylated CpG dinucleotides. For example, as shown in FIG. 7D, an amplification site includes one methylated strand 13 and multiple copies of the same unmethylated nucleotide sequence 13′, coupled to the amplification site surface via respective capture primers 11. The amplification sites also include multiple copies of the unmethylated nucleotide sequence 12′, which includes the same nucleotide sequence as 12 in FIG. 7A-C but is now attached to the amplification site surface via respective capture primers 15.

In some embodiments, amplification methods include, but are not limited to, solid-phase amplification. The term “solid-phase amplification” as used herein refers to any polynucleotide amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers are immobilized on the solid support. Solid phase amplification includes, but is not limited to, systems such as arrays, where one primer is anchored to the surface of the array and the other is in free solution; emulsions, where one primer is anchored to a bead and the other is in free solution; and colony formation in solid phase gel matrices, where one primer is anchored to the surface and one is in free solution. In some embodiments, methods that rely on bridge amplification, where both primers are attached to a surface (see, e.g., WO 2000/018957, U.S. Pat. Nos. 7,972,820; 7,790,418 and Adessi et al., Nucleic Acids Research (2000): 28(20): E87, the entire contents of each of which are incorporated by reference herein) are used. In some embodiments, methods are used that rely on kinetic exclusion, where recombinase-facilitated amplification and isothermal conditions amplify the library (U.S. Pat. Nos. 9,309,502, 8,895,249, 8,071,308, the entire contents of each of which are incorporated by reference herein). Methods that rely on kinetic exclusion are referred to as kinetic exclusion amplification (KEA) or kinetic amplification. Amplification reactions can be performed thermally or isothermally.

A composition for amplifying polynucleotides at amplification sites, referred to herein as an “amplification reagent,” is typically capable of rapidly making copies of polynucleotides at amplification sites. An amplification reagent used in a method of the present disclosure will generally include a polymerase and nucleotide triphosphates (NTPs). Any of a variety of polymerases known in the art can be used, but in some embodiments it may be preferable to use a polymerase that is exonuclease negative. Examples of polymerases suitable for use in embodiments of the present disclosure include, but are not limited to, DNA polymerase (such as Klenow fragment, T4 DNA polymerase, Bst (Bacillus stearothermophilus) polymerase), thermostable DNA polymerases (such as Taq, Vent, Deep Vent, Pfu, Tfl, and 9° N DNA polymerases) as well as their genetically modified derivatives (see, for example, U.S. Pat. Nos. 9,677,057, 11,001,816, and U.S. Pub. No. 2020/0131484A1, the entire contents of each of which are incorporated by reference herein). In some embodiments, an amplification reagent can also include recombinase, accessory protein, and single-stranded DNA binding (SSB) protein for recombinase-facilitated amplification (see, for example, U.S. Pat. No. 8,071,308, the entire contents of which are incorporated by reference herein).

Other components of the amplification solution are added consequently to the choice of the polymerase, and they are essentially corresponding to compounds known in the art as being effective to support the activity of each polymerase. The concentration of compounds like dimethyl sulfoxide (DMSO), Bovine Serum Albumin (BSA), poly-ethylene glycol (PEG), Betaine, Triton X-100, denaturant (e.g., formamide), or MgCl₂ is well known in the prior art as being important to have an optimal amplification, and therefore the operator can easily adjust such concentrations for the methods of the present disclosure on the basis of the examples presented hereafter and the knowledge generally available.

The method further includes propagating, at each amplification site, the methylated CpG dinucleotide present on one strand 13 of the clonal population to other members 12′, 13′ of the clonal population. In some examples, an isothermal amplification reaction can be performed by incubating the amplification sites with a reaction mixture under conditions that transfer the methylated CpG dinucleotides of strand 13 to other strands 12′, 13′—that is, to unmethylated strands 12′ otherwise having the sequence of the original template strand 12 or to unmethylated strands 13′ having the sequence of the original complementary strand 13′. In some examples, the conditions include exposing the amplification sites to an enzyme such as DNMT1 and a DNA helicase or recombinase. As used herein, the term “recombinase” is intended to be consistent with its use in the art and include, for example, RecA protein, the T4 UvsX protein, the RB69 bacteriophage UvsX protein, and the like. Examples of these proteins are readily available to the skilled person (U.S. Pat. No. 8,071,308, the entire contents of which are incorporated by reference herein). Examples of formulations that include a helicase protein are described in U.S. Pat. Nos. 7,399,590 and 7,829,284, the entire contents of each of which are incorporated by reference herein.

Propagation of the CpG methylation occurs by many cycles of steps that include (i) hybridization of the complementary single-strand polynucleotides within each amplification site, forming a bridged double-stranded fragment, (ii) transfer of any methylated C of methylated CpG dinucleotides from one strand to the other paired strand by an enzyme such as DNMT1, and (iii) unwinding of the now fully-methylated duplex by either helicase-mediated unwinding or strand invasion by another fragment in the cluster mediated by a recombinase. For example, as shown in FIG. 7E, complementary strands 12′ and 13 shown in operation I anneal to form the double-stranded hemi-methylated structure shown in operation II. The methylation status of CpG dinucleotides on strand 13 is transferred to stand 12′ to convert strand 12′ to 12 as shown in operation III. The double-stranded structure shown in operation III is unwound and the process is repeated until the methylation status of the one methylated single-stranded polynucleotide 13 is propagated through both sub-populations of polynucleotides 12′ and 13′ at the amplification sites, resulting in substantially fully methylated clusters as shown at operation IV.

To facilitate analysis of one of the sub-populations, the capture primers coupling the other one of the sub-populations to the surface may be cleaved to remove that other sub-population from the surface. The cleaving of a nucleotide sequence to permit the optional removal of a specific strand is referred to herein as “linearization.” For example, as shown in FIG. 7F, the sub-population of amplicons 12 have been removed using linearization, leaving the amplicons 13 ready for further analysis in a manner such as described elsewhere herein.

Nonlimiting examples of suitable methods for linearization are described in application number WO 2007/010251, U.S. Pat. Nos. 8,431,348, 8,017,335, 8,765,381, and U.S. Pat. Pub. No. 2019/0309360, the entire contents of each of which are incorporated by reference herein. Any suitable cleavage reaction can be used for linearization. Examples of cleavage reactions include, but are not limited to, enzymatic, chemical, and photochemical. Cleavage can be achieved by, for example, RNase digestion or chemical cleavage of a bond between a deoxyribonucleotide and a ribonucleotide, in which case the cleavage site can include one or more ribonucleotides; chemical reduction of a disulfide linkage with a reducing agent (e.g., TCEP), in which case the cleavage site should include an appropriate disulfide linkage; chemical cleavage of a diol linkage with periodate, in which case the cleavage site should include a diol linkage; and generation of an abasic site and subsequent hydrolysis.

Suitable cleavage techniques for use in the method of the disclosure include, but are not limited to, chemical cleavage, cleavage of an abasic site, cleavage of a ribonucleotide, photochemical cleavage, PCR stoppers, cleavage of a peptide linker, enzymatic digestion with nicking endonuclease. The person of ordinary skill in the art will recognize that use of some conditions described herein, for example heat or alkali, may be undesirable in view of the potential for denaturation of the complementary strand from the shortened capture primer.

In some examples, an abasic site is generated and cleaved. An “abasic site” is defined as a position in a polynucleotide from which the base component has been removed. Abasic sites can occur naturally in DNA under physiological conditions by hydrolysis of nucleoside residues, but can also be formed chemically under artificial conditions or by the action of enzymes. Once formed, abasic sites can be cleaved (e.g., by treatment with an endonuclease or other single-stranded cleaving enzyme, exposure to heat or alkali), providing a means for site-specific cleavage the capture primer. In some examples, an abasic site can be created at a pre-determined position of the capture primer and then cleaved by first incorporating deoxyuridine (U) at the pre-determined cleavage site. The enzyme uracil DNA glycosylase (UDG) can then be used to remove the uracil base, generating an abasic site. The strand including the abasic site may then be cleaved at the abasic site by treatment with endonuclease (e.g. EndoIV endonuclease, AP lyase, FPG glycosylase/AP lyase, Endo VIII glycosylase/AP lyase), heat or alkali. Abasic sites may also be generated at non-natural/modified deoxyribonucleotides other than deoxyuridine and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites generated may then be cleaved, typically by treatment with a suitable endonuclease (e.g., EndoIV, AP lyase).

In some examples, the molecules to be cleaved may be exposed to a mixture containing the appropriate glycosylase and one or more suitable endonucleases. In such mixtures the glycosylase and the endonuclease will typically be present in an activity ratio of at least about 2:1. In a particular embodiment, the USER reagent available from New England Biolabs (NEB #M5505S) is used for the creation of a single nucleotide gap at a uracil base in a capture primer. Treatment with endonuclease enzymes gives rise to a 3′-phosphate moiety at the cleavage site, which can be removed with a suitable phosphatase such as alkaline phosphatase.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1-3. (canceled)

4. A method for detecting a first methylated nucleotide in a polynucleotide, the method comprising:
coupling a first quencher to the first methylated nucleotide;
adding fluorescently labeled nucleotides and a polymerase to a primer hybridized to the polynucleotide;
using the first quencher to reduce fluorescence from at least one of the added, fluorescently labeled nucleotides; and
using the reduced fluorescence caused by the first quencher to detect the first methylated nucleotide,
wherein the first methylated nucleotide is selected from the group consisting of 5-methylcytosine (5-mC), 5-hydroxymethylcytosine (5-hmC), and 5-formylcytosine (5-fC);
wherein coupling the first quencher to the first methylated nucleotide comprises:
oxidizing the 5-mC, 5-hmC, or 5-fC to 5-carboxylcytosine (5-caC); and
reacting the 5-carboxyl group of the 5-caC with a first molecule to form a first product,
wherein the first product comprises the first quencher.

5. A method for detecting a first methylated nucleotide in a polynucleotide, the method comprising:
coupling a first quencher to the first methylated nucleotide;
adding fluorescently labeled nucleotides and a polymerase to a primer hybridized to the polynucleotide;
using the first quencher to reduce fluorescence from at least one of the added, fluorescently labeled nucleotides; and
using the reduced fluorescence caused by the first quencher to detect the first methylated nucleotide,
wherein the first methylated nucleotide is selected from the group consisting of 5-methylcytosine (5-mC), 5-hydroxymethylcytosine (5-hmC), and 5-formylcytosine (5-fC);
wherein coupling the first quencher to the first methylated nucleotide comprises:
oxidizing the 5-mC, 5-hmC, or 5-fC to 5-carboxylcytosine (5-caC);
reacting the 5-carboxyl group of the 5-caC with a first molecule to form a first product; and
coupling the first quencher to the first product.

6. The method of claim 4, wherein a ten-eleven translocation (TET) dioxygenase is used to oxidize the 5-mC, 5-hmC, or 5-fC to 5-caC.

7. The method of claim 4, wherein a chemical reagent is used to oxidize the 5-mC, 5-hmC, or 5-fC.

8. The method of claim 4, further comprising activating the 5-carboxyl group of the 5-caC before reacting the 5-carboxyl group with the first molecule.

9. The method of claim 8, wherein the 5-carboxyl group of the 5-caC is activated using 4-(4,6-dimethoxy-1,3,5-triazin-2-yl)-4-methyl-morpholinium chloride (DMTMM) or 1-ethyl-3-(3′-(dimethylamino) propyl) carbodiimide (EDC).

10. The method of claim 8, wherein the first molecule comprises a nucleophile.

11. The method of claim 4, wherein the first molecule comprises an azirine.

12-17. (canceled)

18. A method for detecting a first methylated nucleotide in a polynucleotide, the method comprising:
coupling a first quencher to the first methylated nucleotide;
adding fluorescently labeled nucleotides and a polymerase to a primer hybridized to the polynucleotide;
using the first quencher to reduce fluorescence from at least one of the added, fluorescently labeled nucleotides; and
using the reduced fluorescence caused by the first quencher to detect the first methylated nucleotide,
wherein the first methylated nucleotide is 5-methylcytosine (5-mC); and
wherein coupling the first quencher to the first methylated nucleotide comprises:
oxidizing the 5-mC to 5-hmC; and
reacting the 5-hydroxymethyl group of the 5-hmC with a first molecule to form a first product; and
reacting the first product with a second molecule to couple the first quencher to the first product.

19. The method of claim 18, wherein a ten-eleven translocation (TET) dioxygenase is used to oxidize the 5-mC to the 5-hmC.

20. The method of claim 19, wherein the TET dioxygenase comprises ccTET.

21. The method of claim 18, wherein reacting the hydroxymethyl group of the 5-hmC with the first molecule comprises using Mha.I to couple the first molecule to the hydroxymethyl group.

22. The method of claim 18, wherein the first molecule comprises an aminothiol.

23. The method of claim 18, wherein the second molecule comprises an N-hydroxysuccinimide (NHS) ester, an isocyanate, or an isothiocyanate.

24-28. (canceled)

29. The method of claim 4, wherein the polynucleotide comprises a second methylated nucleotide, the method further comprising:
coupling a second quencher to the second methylated nucleotide;
using the second quencher to reduce fluorescence from at least one of the added, fluorescently labeled nucleotides; and
using the reduced fluorescence caused by the second quencher to detect the second methylated nucleotide.

30. The method of claim 4, wherein the polynucleotide is coupled to a substrate.

31. The method of claim 30, wherein the polynucleotide is within a cluster of polynucleotide amplicons coupled to the substrate.

32. The method of claim 31, wherein the polynucleotide amplicons of the cluster also respectively include first methylated nucleotides, the method further comprising:
coupling the first quencher to the first methylated nucleotides of the respective amplicons;
adding fluorescently labeled nucleotides to primers respectively hybridized to the amplicons;
using the first quencher to reduce fluorescence from at least one of the added, fluorescently labeled nucleotides; and
using the reduced fluorescence caused by the first quencher to detect the first methylated nucleotides in the respective amplicons.

33-49. (canceled)

50. The method of claim 5, wherein a ten-eleven translocation (TET) dioxygenase is used to oxidize the 5-mC, 5-hmC, or 5-fC to 5-caC.

51. The method of claim 5, wherein a chemical reagent is used to oxidize the 5-mC, 5-hmC, or 5-fC.
